(19) United States
(12) Patent Application Publication
(10) Pub. No.: US 2008/0051424 A1
(43) Pub. Date: Feb. 28, 2008

(54) Heterocyclyl-substituted nonadepsipeptides

(76) Inventors: Franz Von Nussbaum, Duesseldorf (DE); Nina Brunner, Essen (DE); Rainer Endermann, Wuppertal (DE); Chantal Fuerstner, Muelheim An Der Ruhr (DE); Elke Hartmann, Wuppertal (DE); Jacques Ragot, Duesseldorf (DE); Guido Schiffer, Wuppertal (DE); Joachim Schuhmacher, Wuppertal (DE); Niels Svenstrup, Velbert (DE); Joachim Telser, Koeln (DE); Sonja Anlauf, Wuppertal (DE); Michael-Alexander Bruening, Berlin (DE)

Correspondence Address: MORRISON & FOERSTER LLP, 12531 HIGH BLUFF DRIVE, SUITE 100, SAN DIEGO, CA, 92130-2040, US

(21) Appl. No.: 11/788,649

(22) Filed: Apr. 20, 2007

Related U.S. Application Data
(63) Continuation of application No. PCT/EP05/10856, filed on Oct. 8, 2005.

(30) Foreign Application Priority Data
Oct. 20, 2004 (DE) 10 2004 051 024.5

(51) Int. Cl. A61K 31/506; A61P 31/04; C07D 239/24

(52) U.S. Cl. 514/275; 540/454

(57) ABSTRACT

The invention relates to nonadepsipeptides and methods for their preparation as well as their use for the production of medicaments for the treatment and/or prophylaxis of diseases, in particular bacterial infectious diseases.

### CROSS-REFERENCE TO RELATED APPLICATIONS

This application is a continuation of pending international application PCT/EP2005/010856, filed Oct. 8, 2005, designating US, which claims priority from German patent application DE 10 2004 051 024.5, filed Oct. 20, 2004. The contents of the above-referenced applications are incorporated herein by this reference in their entirety.

### BACKGROUND OF THE INVENTION

The invention relates to nonadepsipeptides and methods for their preparation, as well as to their use for the production of medicaments for the treatment and/or prophylaxis of diseases, in particular bacterial infectious diseases.

The bacterial cell wall is synthesized by a number of enzymes (cell wall biosynthesis) and is essential for the survival and reproduction of microorganisms. The structure of this macromolecule, as well as the proteins involved in its synthesis, are highly conserved within the bacteria. On account of its essential nature and uniformity, cell wall biosynthesis is an ideal point of attack for novel antibiotics (D. W. Green, The bacterial cell wall as a source of antibacterial targets, Expert Opin. Ther. Targets, 2002, 6, 1-19).

Vancomycin and penicillins are inhibitors of the bacterial cell wall biosynthesis and represent successful examples of the antibiotic potency of this principle of action. They have been employed for a number of decades clinically for the treatment of bacterial infections, especially with Gram-positive pathogens. Owing to the growing occurrence of resistant microorganisms, e.g. methicillin-resistant staphylococci, penicillin-resistant pneumococci and vancomycin-resistant enterococci (F. Baquero, Gram-positive resistance: challenge for the development of new antibiotics, J Antimicrob. Chemother., 1997, 39, Suppl A: 1-6; A. P. Johnson, D. M. Livermore, G. S. Tillotson, Antimicrobial susceptibility of Gram-positive bacteria: what's current, what's anticipated?, J Hosp. Infect., 2001, (49), Suppl A: 3-11) and recently also for the first time vancomycin-resistant staphylococci (B. Goldrick, First reported case of VRSA in the United States, Am. J. Nurs., 2002, 102, 17), these substances are increasingly losing their therapeutic efficacy.

The present invention describes a novel class of cell wall biosynthesis inhibitors without cross resistances to known classes of antibiotics.

The natural product lysobactin and some derivatives are described as having antibacterial activity in U.S. Pat. No. 4,754,018. The isolation and antibacterial activity of lysobactin is also described in EP-A 196 042 and JP 01132600. WO04/099239 describes derivatives of lysobactin having antibacterial activity.

The antibacterial activity of lysobactin and katanosin A is furthermore described in O'Sullivan, J. et al., J. Antibiot. 1988, 41, 1740-1744, Bonner, D. P. et al., J. Antibiot. 1988, 41, 1745-1751, Shoji, J. et al., J. Antibiot. 1988, 41, 713-718 and Tymiak, A. A. et al., J. Org. Chem. 1989, 54, 1149-1157.

### SUMMARY OF THE INVENTION

It is one object of the present invention to provide alternative compounds having comparable or improved antibacterial activity and better tolerability, e.g. lower nephrotoxicity, for the treatment of bacterial diseases in humans and animals.

The invention relates to compounds of formula

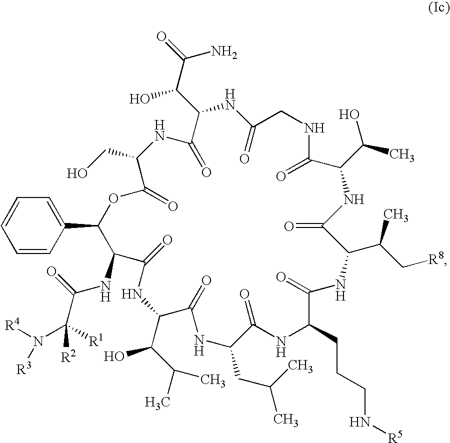

in which

R¹ represents hydrogen, C₁-C₆-alkyl, C₂-C₆-alkenyl, C₃-C₆-cycloalkyl or C₆-C₁₀-aryl,

whereby alkyl, alkenyl, cycloalkyl and aryl can be substituted with 0, 1, 2 or 3 substituents selected independently of one another from the group consisting of halogen, hydroxy, amino, cyano, trimethylsilyl, C₁-C₆-alkyl, C₁-C₆-alkoxy, benzyloxy, C₃-C₆-cycloalkyl, C₆-C₁₀-aryl, 5- to 7-membered heterocyclyl, 5- to 10-membered heteroaryl, C₁-C₆-alkylamino, C₆-C₁₀-arylamino, C₁-C₆-alkylcarbonylamino, C₆-C₁₀-arylcarbonylamino, C₁-C₆-alkylcarbonyl, C₁-C₆-alkoxycarbonyl, C₆-C₁₀-arylcarbonyl and benzyloxycarbonylamino,

wherein cycloalkyl, aryl, heterocyclyl and heteroaryl for their part can be substituted with 0, 1, 2 or 3 substituents selected independently of one another from the group consisting of halogen, hydroxy, amino, cyano, nitro, trifluoromethyl, C₁-C₆-alkyl, C₁-C₆-alkoxy, phenyl and 5- to 7-membered heterocyclyl,

R² represents hydrogen or C₁-C₄-alkyl,

R³ represents C₁-C₆-alkyl, C₃-C₆-cycloalkyl, 5- to 7-membered heterocyclyl, C₆-C₁₀-aryl, 5- or 6-membered heteroaryl, C₁-C₆-alkylcarbonyl, C₁-C₆-alkoxycarbonyl, C₃-C₆-cycloalkylcarbonyl, 5- to 7-membered heterocyclylcarbonyl, C₆-C₁₀-arylcarbonyl, 5- or 6-membered heteroarylcarbonyl or C₁-C₆-alkylaminocarbonyl,

whereby alkyl, cycloalkyl, heterocyclyl, aryl, heteroaryl, alkoxycarbonyl, cycloalkylcarbonyl, heterocyclylcarbonyl, arylcarbonyl, heteroarylcarbonyl and alkylaminocarbonyl can be substituted with 0, 1, 2 or 3 substituents selected independently of one another from the group consisting of halogen, hydroxy, amino, C₁-C₆-alkylamino and phenyl,

and

whereby alkylcarbonyl is substituted with a substituent amino or C₁-C₆-alkylamino,

and

whereby alkylcarbonyl can be substituted with a further 0, 1 or 2 substituents selected independently of one another from the group consisting of halogen, hydroxy, trimethylsilyl, C₁-C₆-alkoxy, C₁-C₆-alkylthio, benzyloxy, C₃-C₆-cycloalkyl, phenyl, naphthyl, 5- to 10-membered heteroaryl, C₁-C₆-alkylcarbonylamino, C₁-C₆-alkoxycarbonylamino, C₆-C₁₀-arylcarbonylamino, C₆-C₁₀-arylcarbonyloxy, benzyloxycarbonyl and benzyloxycarbonylamino,

wherein phenyl and heteroaryl for their part can be substituted with 0, 1, 2 or 3 substituents selected independently of one another from the group consisting of halogen, hydroxy, nitro, C₁-C₆-alkyl, C₁-C₆-alkoxy and phenyl,

R⁴ represents hydrogen, C₁-C₄-alkyl, cyclopropyl or cyclopropylmethyl,

R⁵ represents a group of formula

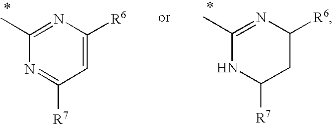

whereby
is the linkage site to the nitrogen atom,

R⁶ and R⁷ independently of one another represent C₁-C₆-alkyl or trifluoromethyl,

R⁸ represents hydrogen or methyl,

and their salts, their solvates and the solvates of their salts.

Compounds of the invention are the compounds of formula (I) and (Ic) and their salts, solvates, solvates of the salts and prodrugs, the compounds of the formulae mentioned below encompassed by formula (I) and (Ic) and their salts, solvates, solvates of the salts and prodrugs, and the compounds mentioned below as exemplary embodiments, encompassed by formula (I) and (Ic), and their salts, solvates, solvates of the salts and prodrugs, insofar as the compounds subsequently mentioned, encompassed by formula (I) and (Ic), are not already salts, solvates, solvates of the salts and prodrugs.

Depending on their structure, the compounds of the invention can exist in stereoisomeric forms (enantiomers, diastereomers). The invention therefore relates to the enantiomers or diastereomers and their respective mixtures. The stereoisomerically uniform constituents can be isolated in a known manner from such mixtures of enantiomers and/or diastereomers.

Where the compounds of the invention can occur in tautomeric forms, the present invention comprises all tautomeric forms.

Salts preferred for the purpose of the present invention are physiologically acceptable salts of the compounds of the invention. However, mixed salts or salts which are not suitable for pharmaceutical applications themselves but can be used, for example, for the isolation or purification of the compounds of the invention are also included.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of usual bases, such as, by way of example and preferably, alkali metal salts (e.g. sodium and potassium salts), alkaline earth metal salts (e.g. calcium and magnesium salts) and ammonium salts, derived from ammonia or organic amines having 1 to 16 C atoms, such as, by way of example and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenerdiamine and N-methylpiperidine.

Solvates for the purpose of the invention refer to those forms of the compounds of the invention which form a complex in the solid or liquid state by coordination with solvent molecules. Hydrates are a special form of solvates in which coordination takes place with water.

For the purpose of the present invention, the substituents have the following meaning unless specified otherwise:

Alkyl per se and “alk” and “alkyl” in alkoxy, alkylamino, alkylthio, alkylcarbonyl, alkoxycarbonyl, alkylaminocarbonyl, alkylcarbonylamino and alkoxycarbonylamino represents a linear or branched alkyl radical normally having 1 to 6, preferably 1 to 4, particularly preferably 1 to 3 carbon atoms, by way of example and preferably methyl, ethyl, n-propyl, isopropyl, tert-butyl, 2,2-dimethylprop-1-yl, 2,2-dimethylbut-1-yl, n-pentyl and n-hexyl.

Alkoxy by way of example and preferably represents methoxy, ethoxy, n-propoxy, isopropoxy, tert-butoxy, n-pentoxy and n-hexoxy.

Alkylthio by way of example and preferably represents methylthio, ethylthio, n-propylthio, isopropylthio, tert-butylthio, n-pentylthio and n-hexylthio.

Alkenyl represents a straight-chain or branched alkenyl radical having 2 to 6 carbon atoms. A straight-chain or branched alkenyl radical having 2 to 4, particularly preferably having 2 to 3 carbon atoms, is preferred. For example and preferably, the following may be mentioned: vinyl, allyl, n-prop-1-en-1-yl, n-but-2-en-1-yl, 2-methylprop-1-en-1-yl and 2-methylprop-2-en-1-yl.

Alkylamino represents an alkylamino radical having one or two (chosen independently of one another) alkyl substituents, by way of example and preferably methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino. C₁-C₃-Alkyl-amino, for example, represents a monoalkylamino radical having 1 to 3 carbon atoms or a dialkylamino radical having 1 to 3 carbon atoms each per alkyl substituent.

Arylamino represents an aryl substituent bonded via an amino group, with a further substituent optionally being bonded to the amino group, such as, for example, aryl or alkyl, by way of example and preferably phenylamino, naphthylamino, phenylmethylamino or diphenyl-amino.

Alkylcarbonyl represents, by way of example and preferably, methylcarbonyl, ethyl-carbonyl, n-propylcarbonyl, isopropylcarbonyl, tert-butylcarbonyl, n-pentylcarbonyl and n-hexylcarbonyl.

Alkoxycarbonyl represents, by way of example and preferably, methoxycarbonyl, ethoxycarbonyl, n-propoxycarbonyl, isopropoxycarbonyl, tert-butoxycarbonyl, n-pentoxycarbonyl and n-hexoxycarbonyl.

Alkoxycarbonylamino represents, by way of example and preferably, methoxycarbonylamino, ethoxycarbonylamino, n-propoxycarbonylamino, isopropoxycarbonylamino, tert-butoxycarbonylamino, n-pentoxycarbonylamino and n-hexoxycarbonylamino.

Cycloalkylcarbonyl represents a cycloalkyl substituent bonded via a carbonyl group, by way of example and preferably, cyclopropylcarbonyl, cyclobutylcarbonyl, cyclopentylcarbonyl and cyclohexylcarbonyl.

Heterocyclylcarbonyl represents a heterocyclyl substituent bonded via a carbonyl group, by way of example and preferably tetrahydrofuranylcarbonyl, pyrrolidinylcarbonyl, pyrrolinylcarbonyl, piperidinylcarbonyl, tetrahydropyranylcarbonyl, piperazinylcarbonyl, morpholinylcarbonyl and perhydroazepinylcarbonyl.

Arylcarbonyl represents an aryl substituent bonded via a carbonyl group, by way of example and preferably phenylcarbonyl, naphthylcarbonyl and phenanthrenylcarbonyl.

Heteroarylcarbonyl represents a heteroaryl substituent bonded via a carbonyl group, by way of example and preferably thienylcarbonyl, furylcarbonyl, pyrrolylcarbonyl, thiazolylcarbonyl, oxazolylcarbonyl, imidazolylcarbonyl, pyridylcarbonyl, pyrimidylcarbonyl, pyridazinylcarbonyl, indolylcarbonyl, indazolylcarbonyl, benzofuranylcarbonyl, benzothiophenylcarbonyl, quinolinylcarbonyl and isoquinolinylcarbonyl.

Alkylcarbonylamino represents, by way of example and preferably, methylcarbonylamino, ethylcarbonylamino, n-propylcarbonylamino, isopropylcarbonylamino, tert-butylcarbonylamino, n-pentylcarbonylamino and n-hexylcarbonylamino.

Arylcarbonylamino represents, by way of example and preferably, phenylcarbonylamino, naphthylcarbonylamino and phenanthrenylcarbonylamino.

Arylcarbonyloxy represents, by way of example and preferably, phenylcarbonyloxy, naphthylcarbonyloxy and phenanthrenylcarbonyloxy.

Alkylaminocarbonyl represents an alkylaminocarbonyl radical having one or two (chosen independently of one another) alkyl substituents, by way of example and preferably methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-tert-butyl-N-methylaminocarbonyl, N-ethyl-N-n-pentylaminocarbonyl and N-n-hexyl-N-methylaminocarbonyl. C₁-C₃-Alkylaminocarbonyl represents, for example, a monoalkylaminocarbonyl radical having 1 to 3 carbon atoms or a dialkylaminocarbonyl radical having 1 to 3 carbon atoms each per alkyl substituent.

Cycloalkyl represents a cycloalkyl group normally having 3 to 6 carbon atoms, by way of example and preferably cyclopropyl, cyclobutyl, cyclopentyl and cyclohexyl.

Aryl represents a mono- to tricyclic aromatic, carbocyclic radical normally having 6 to 14 carbon atoms; by way of example and preferably phenyl, naphthyl and phenanthrenyl.

Heterocyclyl represents a mono- or polycyclic, preferably mono- or bicyclic, heterocyclic radical normally having 5 to 7 ring atoms and up to 3, preferably up to 2, hetero-atoms and/or hetero groups from the series N, O, S, SO, SO₂. The heterocyclyl radicals can be saturated or partly unsaturated. 5- to 7-membered, monocyclic saturated heterocyclyl radicals having up to two heteroatoms from the series O, N and S are preferred, such as, by way of example and preferably, tetrahydrofuranyl, pyrrolidinyl, pyrrolinyl, piperidinyl, tetrahydropyranyl, piperazinyl, morpholinyl and perhydroazepinyl.

Heteroaryl represents an aromatic, mono- or bicyclic radical normally having 5 to 10, preferably 5 to 6 ring atoms and up to 5, preferably up to 4 heteroatoms from the series S, O and N, by way of example and preferably thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl and isoquinolinyl.

Halogen represents fluorine, chlorine, bromine and iodine, preferably fluorine and chlorine.

### BRIEF DESCRIPTION OF THE DRAWINGS

### Description of the Figures

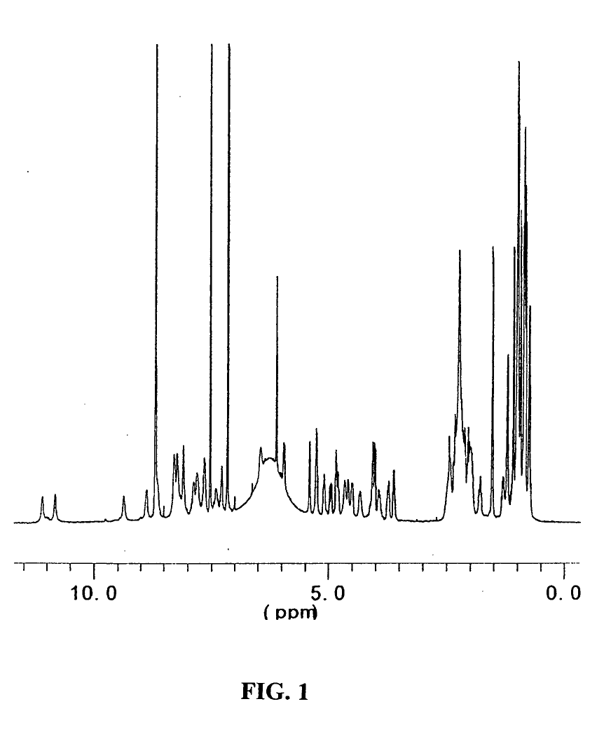

FIG. 1: ¹H-NMR of N^{ω.6},N^{ω′.6}-(pent[2]en[2]yl[4]ylidene)lysobactin trifluoroacetate (d₅-pyridine, 500 MHz).

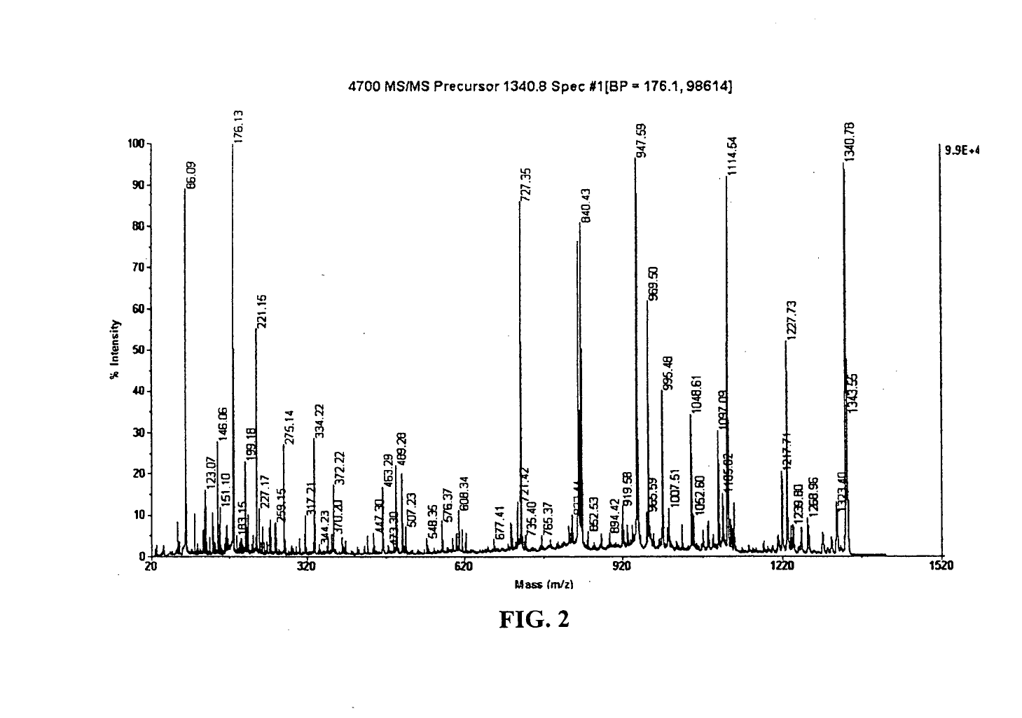

FIG. 2: MALDI-MS/MS-spectrum (positive ions, precursor ion m/z 1340.8) of N^{ω.6},N^{107 ′.6}-(pent[2]en[2]yl[4]ylidene) lysobactin trifluoroacetate.

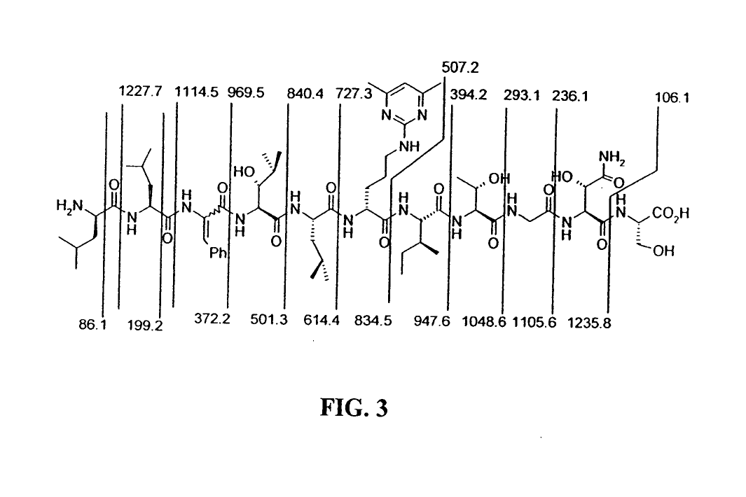

FIG. 3: Fragments of the MALDI-MS/MS spectrum (positive ions, precursor ion m/z 1340.8) of N^{ω.6},N^{ω′.6}-(pent[2]en[2]yl[4]ylidene)lysobactin trifluoroacetate.

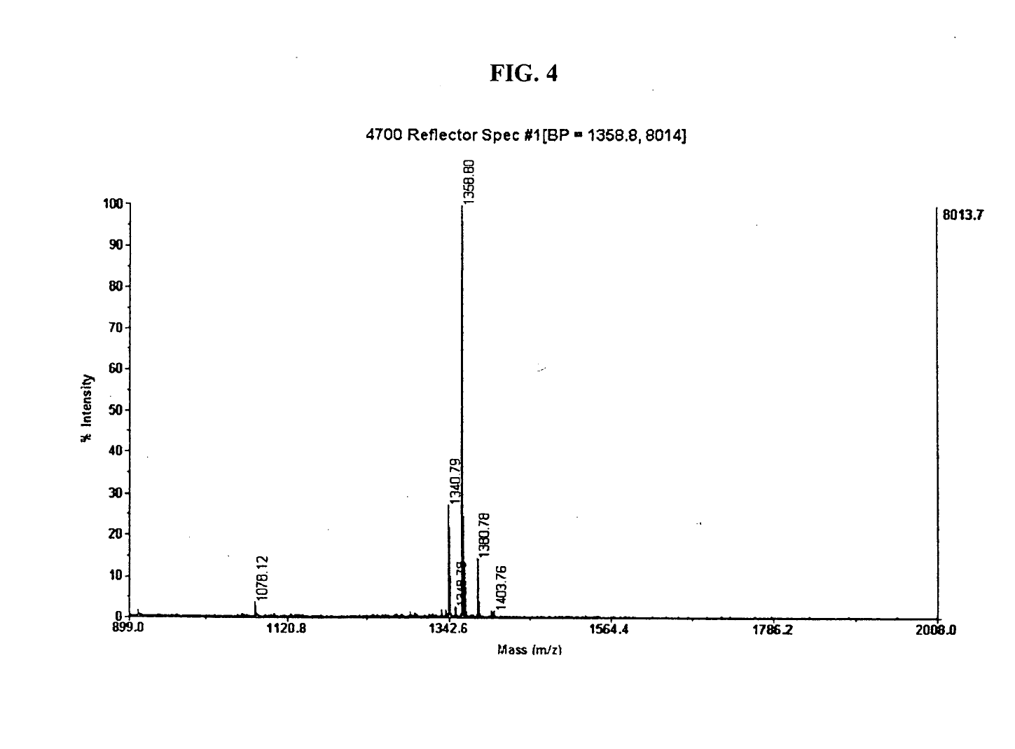

FIG. 4: MALDI-MS spectrum (positive ions) of hydrolytically ring-opened N^{ω.6},N^{ω′.6}-(pent[2]en[2]yl[4]ylidene)lysobactin trifluoroacetate.

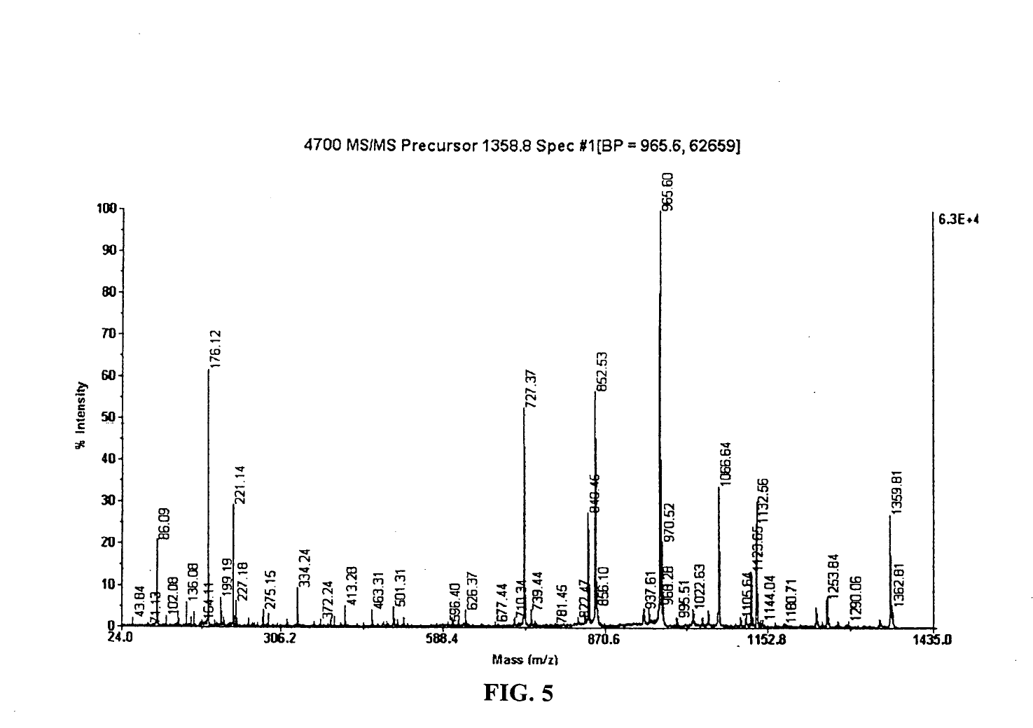

FIG. 5: MALDI-MS/MS-spectrum (positive ions, precursor ion m/z 1358.8) of hydrolytically ring-opened N^{ω.6},N^{ω′.6}-(pent[2]en[2]yl[4]ylidene)lysobactin trifluoroacetate.

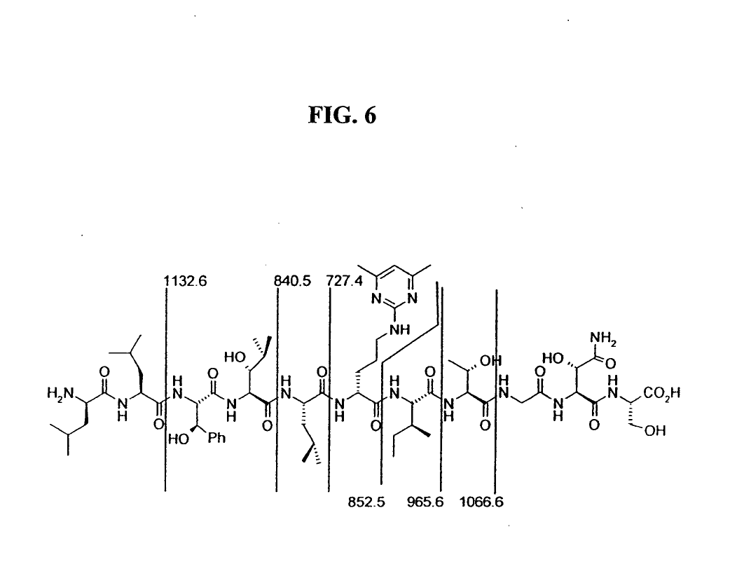

FIG. 6: Fragments of the MALDI-MS/MS spectrum (positive ions, precursor ion m/z 1358.8) of hydrolytically ring-opened N^{ω.6},N^{ω′.6}-(pent[2]en[2]yl[4]ylidene)lysobactin trifluoroacetate.

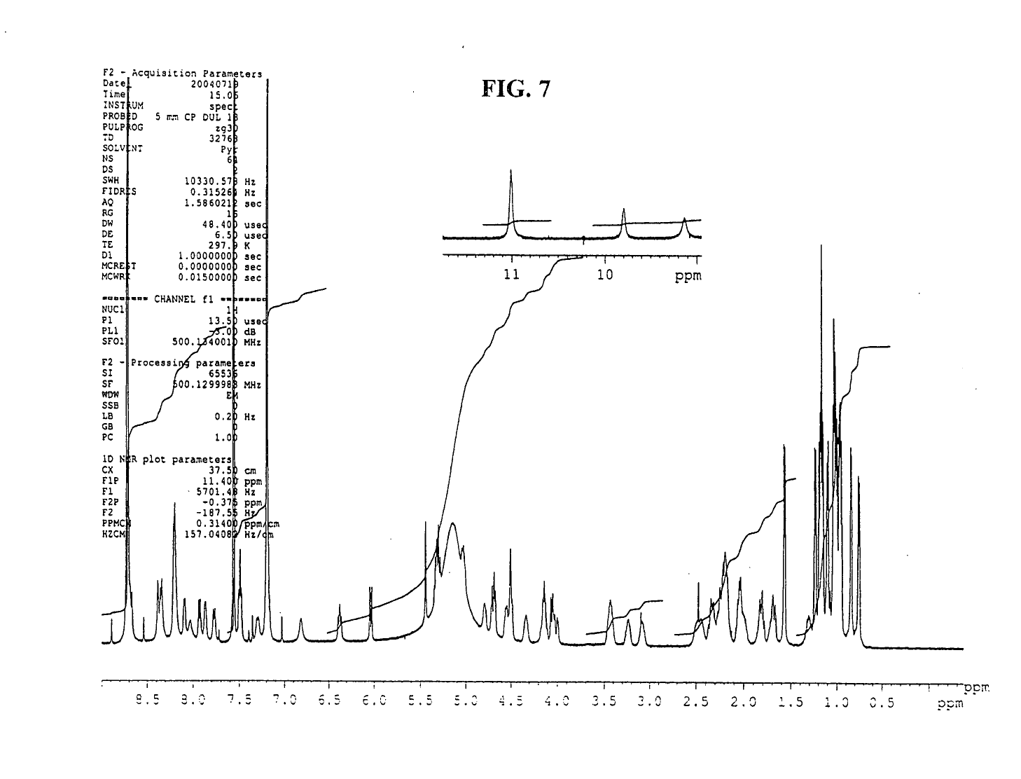

FIG. 7: ¹H-NMR of N^{ω.6},N^{ω′.6}-(Pentane[2,4]diyl)lysobactin trifluoroacetate (d₅-pyridine, 500 MHz).

Preferred compounds in the context of the present invention are those of formula

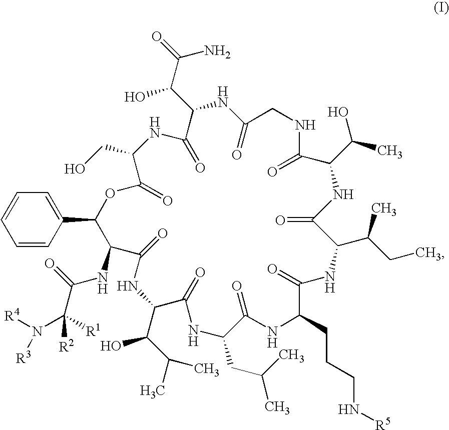

in which

R¹ represents hydrogen, C₁-C₆-alkyl, C₂-C₆-alkenyl, C₃-C₆-cycloalkyl or C₆-C₁₀-aryl,

whereby alkyl, alkenyl, cycloalkyl and aryl can be substituted with 0, 1, 2 or 3 substituents selected independently of one another from the group consisting of halogen, hydroxy, amino, cyano, trimethylsilyl, C₁-C₆-alkyl, C₁-C₆-alkoxy, benzyloxy, C₃-C₆-cycloalkyl, C₆-C₁₀-aryl, 5- to 7-membered heterocyclyl, 5- to 10-membered heteroaryl, C₁-C₆-alkylamino, C₆-C₁₀-arylamino, C₁-C₆-alkylcarbonylamino, C₆-C₁₀-arylcarbonylamino, C₁-C₆-alkylcarbonyl, C₁-C₆-alkoxycarbonyl, C₆-C₁₀-arylcarbonyl and benzyloxycarbonylamino,

wherein cycloalkyl, aryl, heterocyclyl and heteroaryl for their part can be substituted with 0, 1, 2 or 3 substituents selected independently of one another from the group consisting of halogen, hydroxy, amino, cyano, nitro, trifluoromethyl, C₁-C₆-alkyl, C₁-C₆-alkoxy, phenyl and 5- to 7-membered heterocyclyl,

R² represents hydrogen or C₁-C₄-alkyl,

R³ represents C₁-C₆-alkyl, C₃-C₆-cycloalkyl, 5- to 7-membered heterocyclyl, C₆-C₁₀-aryl, 5- or 6-membered heteroaryl, C₁-C₆-alkylcarbonyl, C₁-C₆-alkoxycarbonyl, C₃-C₆-cycloalkylcarbonyl, 5- to 7-membered heterocyclylcarbonyl, C₆-C₁₀-arylcarbonyl, 5- or 6-membered heteroarylcarbonyl or C₁-C₆-alkylaminocarbonyl,

whereby alkyl, cycloalkyl, heterocyclyl, aryl, heteroaryl, alkoxycarbonyl, cycloalkyl-carbonyl, heterocyclylcarbonyl, arylcarbonyl, heteroarylcarbonyl and alkylaminocarbonyl can be substituted with 0, 1, 2 or 3 substituents selected independently of one another from the group consisting of halogen, hydroxy, amino, C₁-C₆-alkylamino and phenyl,

and

whereby alkylcarbonyl is substituted with a substituent amino or C₁-C₆-alkylamino,

and

whereby alkylcarbonyl can be substituted with a further 0, 1 or 2 substituents selected independently of one another from the group consisting of halogen, hydroxy, trimethylsilyl, C₁-C₆-alkoxy, C₁-C₆-alkylthio, benzyloxy, C₃-C₆-cycloalkyl, phenyl, naphthyl, 5- to 10-membered heteroaryl, C₁-C₆-alkylcarbonylamino, C₁-C₆-alkoxycarbonylamino, C₆-C₁₀-arylcarbonylamino, C₆-C₁₀-arylcarbonyloxy, benzyloxycarbonyl and benzyloxycarbonylamino,

wherein phenyl and heteroaryl for their part can be substituted with 0, 1, 2 or 3 substituents selected independently of one another from the group consisting of halogen, hydroxy, nitro, C₁-C₆-alkyl, C₁-C₆-alkoxy and phenyl,

R⁴ represents hydrogen, C₁-C₄-alkyl, cyclopropyl or cyclopropylmethyl,

R⁵ represents a group of formula

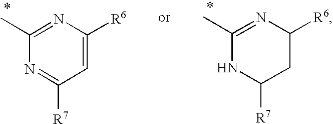

whereby
is the linkage site to the nitrogen atom,
R⁶ and R⁷ independently of one another represent C₁-C₆-alkyl or trifluoromethyl, and their salts, their solvates and the solvates of their salts.

Preferred compounds of formula (I) are also those in which

R¹ represents 2-methylprop-1-yl, 2,2-dimethylprop-1-yl, 2,2-dimethylbut-1-yl, 1-trimethylsilylmethyl, 2-trimethylsilyleth-1-yl, 1-hydroxy-2-methylprop-1-yl, 1-hydroxy-2,2-dimethylprop-1-yl, 1-hydroxy-2,2-dimethylbut-1-yl, 1-hydroxy-2-ethyl-2-methylbut-1-yl, 1-hydroxy-2,2-diethylbut-1-yl, phenylmethyl, 1-hydroxy-1-phenylmethyl, 2-pyridylmethyl or 3-pyridylmethyl,

whereby 2-pyridylmethyl or 3-pyridylmethyl can be substituted with 0, 1, 2 or 3 substituents selected independently of one another from the group consisting of hydroxy, amino, trifluoromethyl, methyl, methoxy and morpholinyl,

R² represents hydrogen,

R³ represents 1-amino-3-methylbut-1-ylcarbonyl, 1-amino-3,3-dimethylbut-1-ylcarbonyl or 1-amino-2-trimethylsilyleth-1-ylcarbonyl,

R⁴ represents hydrogen,

R⁵ represents a group of formula

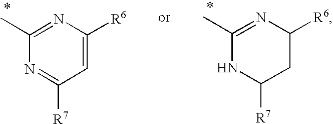

whereby
is the linkage site to the nitrogen atom,
R⁶ and R⁷ independently of one another represent C₁-C₆-alkyl or trifluoromethyl, and their salts, their solvates and the solvates of their salts.

Preferred compounds of formula (I) are also those in which

R¹ represents 2,2-dimethylprop-1-yl, 1-trimethylsilylmethyl or 3-pyridylmethyl, whereby 3-pyridylmethyl can be substituted with a substituent trifluoromethyl,

R² represents hydrogen,

R³ represents 1-amino-3,3-dimethylbut-1-ylcarbonyl or 1-amino-2-trimethylsilyleth-1-ylcarbonyl,

R⁴ represents hydrogen,

R⁵ represents a group of formula

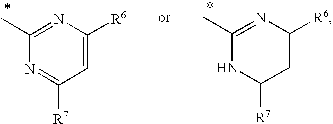

whereby
is the linkage site to the nitrogen atom,
R⁶ and R⁷ independently of one another represent C₁-C₆-alkyl or trifluoromethyl, and their salts, their solvates and the solvates of their salts.

Preferred compounds of formula (I) are also those in which

R¹ represents 2-methylprop-1-yl,

R² represents hydrogen,

R³ represents 1-amino-3-methylbut-1-ylcarbonyl,

R⁴ represents hydrogen,

R⁵ represents a group of formula

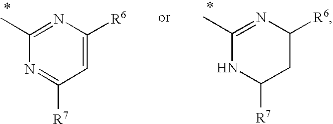

whereby
is the linkage site to the nitrogen atom,

R⁶ and R⁷ independently of one another represent C₁-C₆-alkyl, and their salts, their solvates and the solvates of their salts.

Preferred compounds of formula (I) are also those in which

R¹ represents 2,2-dimethylprop-1-yl,

R² represents hydrogen,

R³ represents 1-amino-3,3-dimethylbut-1-ylcarbonyl,

R⁴ represents hydrogen,

R⁵ represents a group of formula

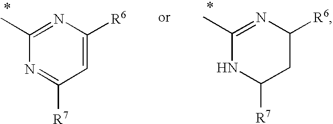

whereby
is the linkage site to the nitrogen atom,

R⁶ and R⁷ independently of one another represent C₁-C₆-alkyl, and their salts, their solvates and the solvates of their salts.

Preferred compounds of formula (I) are also those in which

R¹ represents 2-methylprop-1-yl, 2,2-dimethylprop-1-yl, 2,2-dimethylbut-1-yl, 2-trimethylsilyleth-1-yl, 1-hydroxy-2-methylprop-1-yl, 1-hydroxy-2,2-dimethylprop-1-yl, 1-hydroxy-2,2-dimethylbut-1-yl, 1-hydroxy-2-ethyl-2-methylbut-1-yl, 1-hydroxy-2,2-diethylbut-1-yl, phenylmethyl, 1-hydroxy-1-phenylmethyl, 2-pyridylmethyl or 3-pyridylmethyl,

whereby 2-pyridylmethyl or 3-pyridylmethyl can be substituted with 0, 1, 2 or 3 substituents selected independently of one another from the group consisting of hydroxy, amino, trifluoromethyl, methyl, methoxy and morpholinyl.

Preferred compounds of formula (I) are also those in which the stereocentre derived from an amino acid in R³ has the D configuration.

Preferred compounds of formula (I) are also those in which R¹ is 2,2-dimethylprop-1-yl and R² is hydrogen.

Preferred compounds of formula (I) are also those in which R³ represents 1-amino-3,3-dimethylbut-1-ylcarbonyl and R⁴ represents hydrogen.

The radical definitions indicated in detail in the respective combinations or preferred combinations of radicals are arbitrarily also replaced by radical definitions of a different combination independently of the respective combinations of the radicals indicated.

Combinations of two or more of the abovementioned preferred ranges are also very particularly preferred.

The invention furthermore relates to a method for preparing the compounds of the formulae (Ic), whereby, according to method

[A], compounds of formula

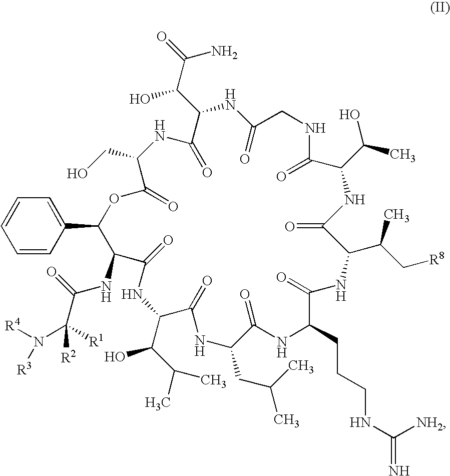

in which R¹, R², R³, R⁴ and R⁸ have the meaning indicated above,

are reacted with compounds of formula

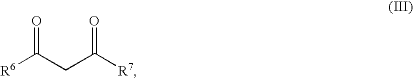

in which R⁶ and R⁷ have the meaning indicated above,

to give compounds of formula

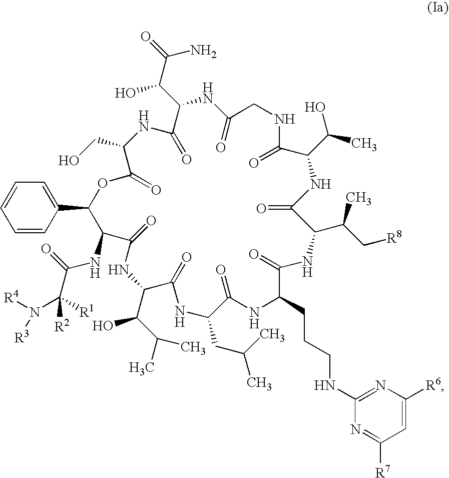

in which R¹, R², R³, R⁴, R⁶, R⁷ and R⁸ have the meaning indicated above,

or

[B] compounds of formula (Ia) are reacted with a reducing agent to give compounds of formula

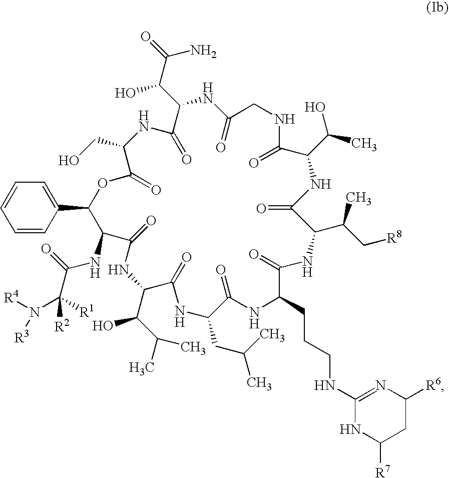

in which R¹, R², R³, R⁴, R⁶, R⁷ and R⁸ have the meaning indicated above.

The compounds of formula (Ic) themselves consist of the compounds of formulae (Ia) and (Ib).

Free amino groups in the radicals R¹, R², R³ and R⁴ are protected before the reaction, where appropriate, according to methods known to the person skilled in the art, for example with a Boc protecting group or a Z protecting group, which is removed again after the reaction.

The compounds of formula (II) are known or can be prepared by reacting the compound of formula

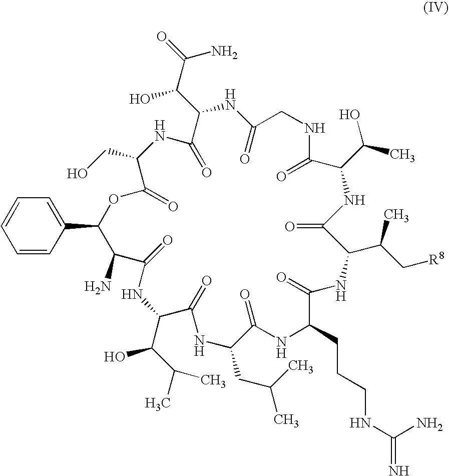

in which

R⁸ has the meaning indicated above,

with compounds of formula

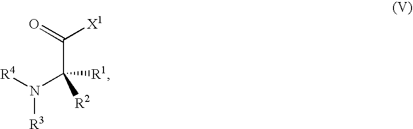

in which

R¹, R², R³ and R⁴ have the meaning indicated above, and

X¹ represents halogen, preferably bromine, chlorine or fluorine, or hydroxy.

If X¹ is halogen, the reaction generally takes place in inert solvents, where appropriate in the presence of a base, preferably in a temperature range from −30° C. to 150° C. under atmospheric pressure.

Inert solvents are, for example, tetrahydrofuran, methylene chloride, acetonitrile, pyridine, dioxane or dimethylformamide. Preferred inert solvents are tetrahydrofuran or methylene chloride.

Bases are, for example, triethylamine, diisopropylethylamine or N-methyl-morpholine; diisopropylethylamine is preferred.

If X¹ is hydroxy, the reaction generally takes place in inert solvents, in the presence of a dehydrating reagent, where appropriate in the presence of a base, preferably in a temperature range from −30° C. to 50° C. under atmospheric pressure.

Inert solvents are, for example, halohydrocarbons such as dichloromethane or trichloromethane, hydrocarbons such as benzene, nitromethane, dioxane, dimethylformamide or acetonitrile. It is likewise possible to employ mixtures of the solvents. Dichloromethane or dimethylformamide are particularly preferred.

Suitable dehydrating reagents hereby are, for example, carbodiimides such as, for example, N,N′-diethyl-, N,N,′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexyl-carbodiimide-N′-propyloxymethylpolystyrene (PS carbodiimide) or carbonyl compounds such as carbonyldiimidazole, or 1,2-oxazolium compounds such as 2-ethyl-5-phenyl-1,2-oxazolium-3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutylchloroformate, or bis-(2-oxo-3-oxazolidinyl)phosphoryl chloride or benzotriazolyloxytri-(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or N-hydroxysuccinimide, or mixtures of these, with bases.

Bases are, for example, alkali metal carbonates, such as, for example, sodium or potassium carbonate, or hydrogencarbonate, or organic bases such as trialkylamines, e.g. triethylamine, N-methylmorpholine, N-methylpiperidine, 4-dimethylaminopyridine or diisopropylethylamine.

Preferably, the condensation is carried out using HATU or using EDC in the presence of HOBt.

The compounds of formula (V) optionally bear protecting groups, so that in these cases the reaction of compounds of formula (IV) with compounds of formula (V) is followed by the removal of the protecting groups using, for example, trifluoroacetic acid according to the methods known to the person skilled in the art.

The compound of formula (IV) can be synthesized from lysobactin (Example 1A) by double Edmann degradation.

The compounds of formulae (III) and (V) are known or can be synthesized from the corresponding starting materials by known processes.

The preparation of the compounds of the invention can be illustrated by the following synthesis scheme.

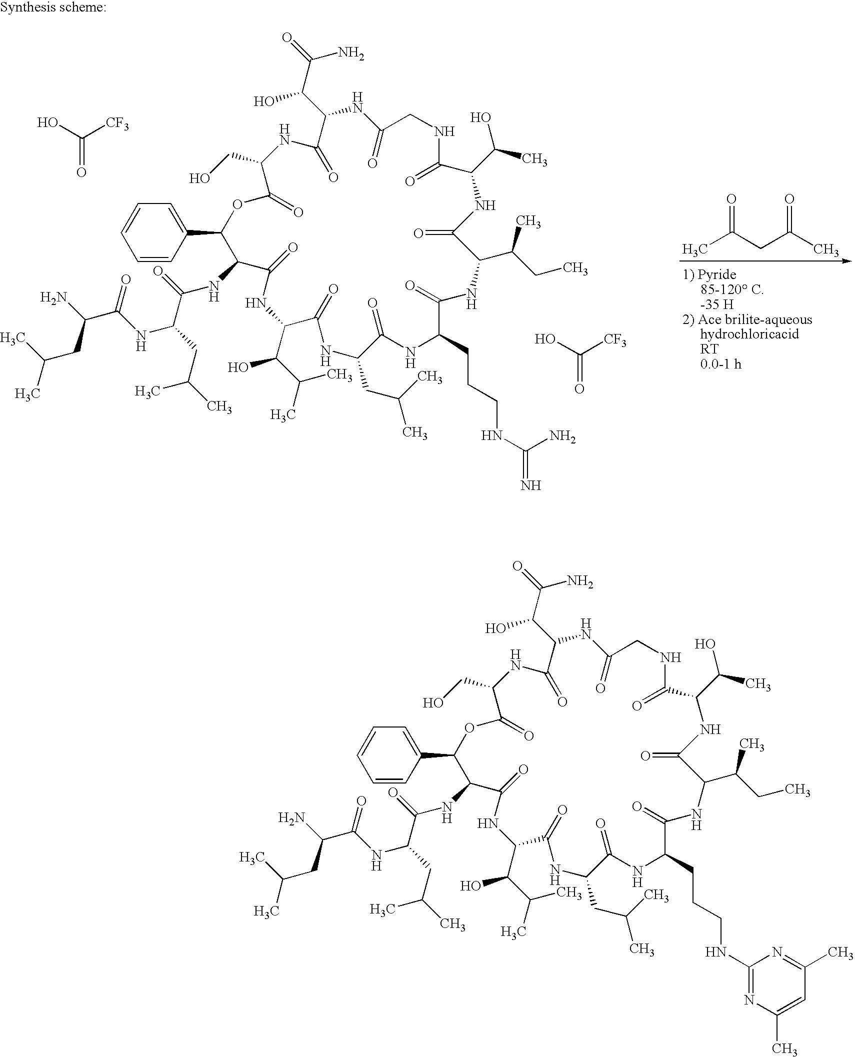

The compounds of the invention show a valuable spectrum of pharmacological activity which could not have been predicted. They show an antibacterial activity.

They are therefore suitable for use as medicaments for the treatment and/or prophylaxis of diseases in humans and animals.

The compounds of the invention are distinguished by a lower nephrotoxicity compared to lysobactin.

The nonadepsipeptides described act as inhibitors of the bacterial cell wall biosynthesis.

The preparations of the invention are particularly effective against bacteria and bacteria-like microorganisms. They are therefore particularly suitable for the prophylaxis and chemotherapy of local and systemic infections caused by these pathogens in human and veterinary medicine.

In principle, the preparations of the invention can be used against all bacteria and bacteria-like microorganisms which possess a bacterial cell wall (Murein sacculus) or the corresponding enzyme systems, for example by the following pathogens or by mixtures of the following pathogens:

Gram-negative cocci (Neisseria gonorrhoeae) as well as Gram-negative rods such as Enterobacteriaceae, e.g. Escherichia coli, Haemophilus influenzae, Pseudomonas, Klebsiella, Citrobacter (C. freundii, C. divernis), Salmonella and Shigella; furthermore Enterobacter (E. aerogenes, E. agglomerans), Hafnia, Serratia (S. marcescens), Providencia, Yersinia, as well as the genus Acinetobacter, Branhamella and Chlamydia. Moreover, the antibacterial spectrum includes strictly anaerobic bacteria such as, for example, Bacteroides fragilis, representatives of the genus Peptococcus, Peptostreptococcus as well as the genus Clostridium; furthermore Mycobacteria, e.g. M. tuberculosus. The compounds of the invention show a particularly pronounced effect on Gram-positive cocci, e.g. staphylococci (S. aureus, S. epidermidis, S. haemolyticus, S. carnosus), enterococci (E. faecalis, E. faecium) and streptococci (S. agalactiae, S. pneumoniae, S. pyogenes).

The above list of pathogens is to be interpreted only by way of example and in no way as restrictive. Diseases which may be mentioned which are caused by the pathogens mentioned or mixed infections and can be prevented, ameliorated or cured by the preparations of the invention are, for example:

Infectious diseases in humans such as, for example, uncomplicated and complicated urinary tract infections, uncomplicated skin and superficial infections, complicated skin and soft tissue infections, pneumonia acquired in hospital and as an outpatient, nosocomial pneumonia, acute exacerbations and secondary bacterial infections of chronic bronchitis, acute otitis media, acute sinusitis, streptococcal pharyngitis, bacterial meningitis, uncomplicated gonococcal and non-gonococcal urethritis/cervicitis, acute prostatitis, endocarditis, uncomplicated and complicated intra-abdominal infections, gynaecological infections, pelvic inflammatory disease, bacterial vaginosis, acute and chronic osteomyelitis, acute bacterial arthritis, empirical therapy in febrile neutropenic patients, furthermore bacteraemias, MRSA infections, acute infectious diarrhoea, Helicobacter pylori infections, postoperative infections, odontogenic infections, opthalmological infections, postoperative infections (including periproctal abscess, wound infections, biliary infections, mastitis and acute appendicitis), cystic fibrosis and bronchiectasis.

Apart from in humans, bacterial infections can also be treated in other species. Examples which may be mentioned are:

Pigs: diarrhoea, enterotoxaemia, sepsis, dysentery, salmonellosis, metritis-mastitis-agalactiae syndrome, mastitis;

Ruminants (cattle, sheep, goats): diarrhoea, sepsis, bronchopneumonia, salmonellosis, pasteurellosis, genital infections;

Horses: bronchopneumonia, joint-ill, puerperal and postpuerperal infections, salmonellosis;

Dogs and cats: bronchopneumonia, diarrhoea, dermatitis, otitis, urinary tract infections, prostatitis;

Poultry (chickens, turkeys, quails, pigeons, ornamental birds and others): E. Coli infections, chronic respiratory diseases, salmonellosis, pasteurellosis, psittacosis.

It is likewise possible to treat bacterial diseases in the raising and keeping of productive and ornamental fish, the antibacterial spectrum thereby extending beyond the previously mentioned pathogens to further pathogens such as, for example, Pasteurella, Brucella, Campylobacter, Listeria, Erysipelothris, Corynebacteria, Borellia, Treponema, Nocardia, Rikettsia, Yersinia.

The present invention further relates to the use of the compounds of the invention for the treatment and/or prophylaxis of diseases, in particular of bacterial infectious diseases.

The present invention further relates to the use of the compounds of the invention for the treatment and/or prophylaxis of diseases, in particular the aforementioned diseases.

The present invention further relates to the use of the compounds of the invention for the production of a medicament for the treatment and/or prophylaxis of diseases, in particular the aforementioned diseases.

The compounds of the invention are preferably used for the production of medicaments which are suitable for the prophylaxis and/or treatment of bacterial diseases.

The present invention further relates to methods for the treatment and/or prophylaxis of diseases, in particular the aforementioned diseases, using an antibacterially effective amount of the compounds of the invention.

The present invention further relates to medicaments, comprising at least one compound of the invention and at least one or more further active compounds, in particular for the treatment and/or prophylaxis of the aforementioned diseases. Preferred active compounds for combination are antibacterially active compounds which have a different spectrum of activity, in particular a supplementary spectrum of activity, and/or are synergistic to the compounds of the invention.

The compounds of the invention can act systemically and/or locally. For this purpose, they can be administered in a suitable way, such as, for example, orally, parenterally, pulmonarily, nasally, sublingually, lingually, buccally, rectally, dermally, transdermally, conjunctivally, otically or as an implant or stent.

The compounds of the invention can be administered in administration forms suitable for these administration routes.

Suitable administration for oral administration are forms which function according to the prior art, and deliver the compounds of the invention rapidly and/or in a modified fashion are, and which contain the compounds of the invention in crystalline and/or amorphized and/or dissolved form, such as, for example, tablets (uncoated or coated tablets, for example having enteric coatings which are insoluble or dissolve with a delay, and control the release of the compound of the invention), tablets or films/wafer which disintegrate rapidly in the oral cavity, films/lyophilizates, capsules (for example hard or soft gelatine capsules), sugar-coated tablets, granules, pellets, powders, emulsions, suspensions, aerosols or solutions.

Parenteral administration can take place with avoidance of an absorption step (e.g. intravenous, intraarterial, intracardial, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates, or sterile powders.

Suitable for the other administration routes are, for example, pharmaceutical forms for inhalation (inter alia powder inhalers, nebulizers), nasal drops, solutions, sprays; tablets, films/wafers or capsules, for lingual, sublingual or buccal administration, suppositories, preparations for ears or eyes, vaginal capsules, aqueous suspensions (lotions, shaking mixtures), lipophilic suspensions, ointments, creams, transdermal therapeutic systems (such as, for example, patches), milk, pastes, foams, dusting powder, implants or stents.

The compounds of the invention can be converted into the stated administration forms. This can take place in a manner known per se by mixing with inert, non-toxic, pharmaceutically acceptable excipients. These excipients include, inter alia, carriers (for example microcrystalline cellulose, lactose, mannitol), solvents (e.g. liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and taste and/or odor corrigents.

The present invention furthermore relates to medicaments which contain at least one compound of the invention, usually together with one or more inert, non-toxic, pharmaceutically acceptable excipients, and their use for the aforementioned purposes.

In general, it has proved advantageous on intravenous administration to administer amounts of about 0.001 to 100 mg/kg, preferably about 0.1 to 10 mg/kg of body weight to achieve effective results, and on oral administration the dosage is about 0.01 to 50 mg/kg, preferably 0.5 to 10 mg/kg, of body weight.

It may nevertheless be necessary where appropriate to deviate from the stated amounts, in particular as a function of body weight, route of administration, individual behavior towards the active compound, type of preparation and time or interval over which administration takes place. Thus, in some cases it may be sufficient to make do with less than the aforementioned minimum amount, while in other cases the stated upper limit must be exceeded. In the case of the administration of larger amounts, it can be advisable to divide these into a number of individual administrations over the course of the day.

The percentages in the following Tests and Examples are, unless indicated otherwise, percentages by weight; parts are parts by weight. Solvent ratios, dilution ratios and concentrations of liquid/liquid solutions are in each case based on volume.

### DETAILED DESCRIPTION OF PREFERRED EMBODIMENTS

### A. Examples

Abbreviations
Area (Peak) area
BHI Brain heart infusion
Boc tert-butyloxycarbonyl
br. broad signal (in NMR spectra)
calc. Calculated
conc. Concentrated
d doublet (in NMR spectra)
DCI direct chemical ionization (in MS)
DCM Dichloromethane
DIEA N,N-diisopropylethylamine
DMF N,N-dimethylformamide
DMSO dimethylsulfoxide
EA ethyl acetate (acetic acid ethyl ester)
EDC 1-ethyl-3-(3-dimethylaminopropyl)carbodiimide (also EDCI)
EDC×HCl 1-ethyl-3-(3-dimethylaminopropyl)carbodiimide hydrochloride
EI electron impact ionization (in MS)
ESI electrospray ionization (in MS)
Ex. Example
find. Found
gen. General
h Hour
HATU O-(7-azabenzotriazol-1-yl)-N,N,N′,N′-tetramethyluronium hexa-fluorophosphate
HOBt 1-hydroxybenzotriazole
HPLC high-pressure or high-performance liquid chromatography
HR high resolution
i. V. in vacuo
LC-MS liquid chromatography-coupled mass spectroscopy
LDA lithium diisopropylamide
m middle (in UV and IR spectra)
m multiplet (in NMR spectra)
MALDI matrix-assisted laser desorption/ionization
MIC minimum inhibitory concentration
min minute/minutes
Mp. melting point
MRSA methicillin-resistant Staphylococcus aureus
MS mass spectroscopy
NCCLS National Committee for Clinical Laboratory Standards
neg. Negative
NMM N-methylmorpholine
NMR nuclear magnetic resonance spectroscopy
of th. of theory
p.a. per analysis
Pd—C palladium on carbon
perc. per cent
pos. positive
quant. Quantitative
RP-HPLC reverse phase HPLC
RT room temperature
Rₜ retention time (in HPLC)
s strong (in UV and IR spectra)
s singlet (in NMR spectra)
satd. Saturated
TBTU O-(benzotriazol-1-yl)-N,N,N′,N′-tetramethyluronium tetrafluoroborate
TCTU O-(1H-6-chlorobenzotriazol-1-yl)-1,1,3,3-tetramethyluronium tetrafluoroborate
TFA trifluoroacetic acid
TFE 2,2,2-trifluoroethanol
THF Tetrahydrofuran
TLC thin-layer chromatography
TOF time of flight
UV Ultraviolet
V is visible
VRSA vancomycin-resisant Staphylococcus aureus
w weak (in UV and IR spectra)
Z, Cbz benzyloxycarbonyl

Literature

For the nomenclature of the peptides and cyclodepsipeptides cf.:

1. A Guide to IUPAC Nomenclature of Organic Compounds (Recommendations 1993), 1993, Blackwell Scientific publications.

2. Nomenclature and symbolism for amino acids and peptides. Recommendations 1983. IUPAC-IUB Joint Commission on Biochemical Nomenclature, UK. Biochemical Journal 1984, 219, 345-373. And cited literature.

3. For the nomenclature of nonadepsipeptide derivatives which are derivatized in the amino acid side chains, the IUPAC prefix system for addressing the respective derivatization site is used (IUPAC, Nomenclature and Symbolism for Amino Acids and Peptides, Names and Symbols for Derivatives of Named Peptides, Section 3AA-22, Recommendations 1983-1992). For instance, N^{ω.6}-acetyllysobactin designates a lysobactin acetylated on amino acid 6 (calculated from the N-terminus of the depsipeptide, i.e. here D-Arg), especially on the terminal nitrogen atom.

General Methods LC-MS, HR-MS, HPLC and Gel Chromatography

Method 1 (HPLC): instrument type HPLC: HP 1100 Series; UV DAD column: Zorbax Eclipse XBD-C8 (Agilent), 150 mm×4.6 mm, 5 μm; eluent A: 5 ml of HClO₄/l of water, eluent B: acetonitrile; gradient: 0-1 min 10% B, 1-4 min 10-90% B, 4-5 min 90% B; flow: 2.0 ml/min; oven: 30° C.; UV detection: 210 and 254 nm.

Method 2 (HPLC): column: Kromasil RP-18, 60 mm×2 mm, 3.5 μm; eluent A: 5 ml of HClO₄/l of water, eluent B: acetonitrile; gradient: 0 min 2% B, 0.5 min 2% B, 4.5 min 90% B, 9 min 90% B; flow: 0.75 ml/min; oven: 30° C.; UV detection: 210 nm.

Method 3 (LC-MS): instrument type MS: Micromass ZQ; instrument type HPLC: HP 1100 Series; UV DAD; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow: 0.0 min, 1 ml/min, 2.5 min/3.0 min/4.5 min, 2 ml/min; oven: 50° C.; UV detection: 210 nm.

Method 4 (HPLC): column: Kromasil RP-18, 250 mm×4 mm, 5 μm; eluent A: 5 ml of HClO₄/l of water, eluent B: acetonitrile; gradient: 0 min 5% B, 10 min 95% B; flow: 1 ml/min; oven: 40° C.; UV detection: 210 nm.

Method 5 (HPLC): column: Kromasil RP-18, 250 mm×4 mm, 5 μm; eluent A: 2 ml of HClO₄/l of water, eluent B: acetonitrile; isocratic: 45% B, 55% A; flow: 1 ml/min; oven: 40° C.; UV detection: 210 nm.

Method 6 (Gel chromatography on Sephadex LH-20): Gel chromatography is carried out without pressure on Sephadex LH-20 (Pharmacia). Fractionation (fraction collector ISCO Foxy 200) is carried out according to UV activity (UV detector for 254 nm, Knauer). Column dimensions: 32×7 cm (1000-100 μmol scale); 30×4 cm (100-10 μmol scale); 25×2 cm (10-1 μmol scale).

Method 7 (preparative HPLC): instrument: Gilson Abimed HPLC; UV detector 210 nm; binary pump system; column: Reprosil ODS-3, 5 μm, 250×20 mm; eluent A: 0.2% trifluoroacetic acid in water, eluent B: acetonitrile; flow rate: 25 ml/min; column temperature 40° C.; 0-12 min 35% B.

Method 8 (LC-MS): instrument type MS: Micromass ZQ; instrument type HPLC: HP 1100 Series; UV DAD; column: Phenomenex Synergi 2μ hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow: 0.0 min 1 ml/min, 2.5 min/3.0 min/4.5 min. 2 ml/min; oven: 50° C.; UV detection: 210 nm.

Method 9 (HPLC): instrument type HPLC: HP 1050 Series; UV DAD 1100 Series; column SymmetryPrep™C₁₈, Waters, 50×2.1 mm, 3.5 μm; eluent A: water/0.05% trifluoroacetic acid, eluent B: acetonitrile; gradient: 0-9 min 0-100% B, 9-11 min 100% B, 11-12 min 100-0% B, subsequent regeneration of the chromatography column. Oven: 40° C., flow: 0.4 mL/min, UV detection: 210 nm.

Method 10 (FT-ICR-HR-MS): The mass precision measurements are carried out on a high resolution Apex II Fourier transform ion cyclotron resonance mass spectrometer (Bruker Daltonik GmbH, Bremen), which is equipped with a 7 Tesla magnet, an external electrospray ion source and a Unix-based XMASS data system. The mass resolution is about 40,000 (50% valley definition).

Method 11 (preparative HPLC): instrument: Gilson Abimed HPLC; UV detector 210 nm; binary pump system; column: Kromasil C-18, 5 μm, 100 Å, 250×20 mm; eluent A: 0.2% trifluoroacetic acid in water, eluent B: acetonitrile: flow rate: 25 mL/min; 0 min 20% B, ramp 0-15 min 80% B, ramp, 15-15.1 min 20% B, 15.1-20 min 20% B. For N-butoxycarbonyl-protected substances, the trifluoroacetic acid in the eluent is basically replaced by 0.05% acetic acid.

Method 12 (LC-MS): instrument type MS: Micromass ZQ; instrument type HPLC: Waters Alliance 2790; column: Grom-Sil 120 ODS-4 HE 50×2 mm, 3.0 μm; eluent A: water+500 μl of 50% formic acid/l; eluent B: acetonitrile+500 μl of 50% formic acid/l; gradient: 0.0 min 0% B→0.2 min 0% B→2.9 min 70% B→3.1 min 90% B→4.5 min 90% B; oven: 45° C.; flow: 0.8 ml/min; UV detection: 210 nm.

Method 13 (TOF-HR-ESI-MS): TOF-HR-ESI-MS spectra are measured using a Micromass-LCT mass spectrometer (capillary 3.2 KV, cone 42 V, source: 120° C.). The samples are injected using a syringe pump (Harvard Instrument). Leucine enkephalin is used as standard.

Method 14 (preparative HPLC): instrument: Gilson Abimed HPLC; UV detector 210 nm; binary pump system; column: Nucleodur C18 Gravity, Macherey-Nagel, 5 μm; 250×40 mm; flow: 15-45 mL/min; eluent A: water/0.1% trifluoroacetic acid, eluent B: acetonitrile; gradient: 0-12 min 10% B, 12-20 min 10-35% B, 20-25 min 35-40% B, 25-35 min 40% B, 35-45 min 40-50% B, 45-50 min 50-60% B 100% B, 50-60 min 60-100% B, 60-75 min 100% B, subsequent regeneration of the chromatography column.

Method 15 (MALDI-MS): The MALDI-MS/MS investigations are carried out on a 4700 Proteomics Analyzer (Applied Biosystems, Framingham, Mass., USA) which is equipped with TOF/TOF ion optics and a 200 Hz Nd:YAG laser (355 nm). The quasimolecular ions are accelerated in the ion source using 8 kV, selected using an electrical deflector (MS1), and impacted with argon atoms in an impact cell which is arranged between MS1 and MS2. The resulting fragment ions are re-accelerated using 15 kV and characterized using the second time of flight mass analyser (MS2).

Method 16 (LC-MS): instrument type MS: Micromass ZQ; instrument type HPLC: Waters Alliance 2790; column: Grom-Sil 120 ODS-4 HE 50 mm×2 mm, 3.0 μm; eluent B: acetonitrile+0.05% formic acid, eluent A: water+0.05% formic acid; gradient: 0.0 min 5% B→2.0 min 40% B→4.5 min 90% B→5.5 min 90% B; oven: 45° C.; flow: 0.0 min 0.75 ml/min→4.5 min 0.75 ml/min→5.5 min 1.25 ml/min; UV detection: 210 nm.

Method 17 (preparative HPLC): instrument: Gilson Abimed HPLC; UV detector 210 nm; binary pump system; column: NucleodurC₁₈ Gravity, Macherey-Nagel, 5 μm; 250×21 mm; flow: 20 ml/min; eluent A: water/0.25-0.5% acetic acid, eluent B: acetonitrile; gradient: 0-3 min 5% B, 3-30 min 5-100% B, 30-38 min 100% B, subsequent regeneration of the chromatography column.

Method 18 (NMR, Quantitative TFA analysis/Absolute contents): A dissolved fluorine-containing organic substance and a calibration substance (e.g. 1,4-dibromotetrafluorobenzene) are weighed, a suitable solvent is added and subsequently a ¹⁹F-NMR spectrum of the sample is recorded (376 MHz). The necessary integrals of the test substance and of the calibration substance are determined from the NMR spectrum. The content of fluorine (or TFA) is determined from this.

Method 19 (MALDI-MS): The MALDI-MS/MS investigations are carried out on a 4700 Proteomics Analyzer (Applied Biosystems, Framingham, Mass., USA) which is equipped with TOF/TOF ion optics and a 200 Hz Nd:YAG laser (355 nm). The quasimolecular ions are accelerated in the ion source using 8 kV, selected using an electrical deflector (MS1), and impacted with argon atoms in an impact cell which is arranged between MS1 and MS2. The resulting fragment ions are re-accelerated using 15 kV and characterized using the second time of flight mass analyser (MS2).

Method 20 (FT-ICR-HR-MS): The mass precision measurements are carried out on a high resolution Apex II Fourier transform ion cyclotron resonance mass spectrometer (Bruker Daltonik GmbH, Bremen) which is equipped with a 7 Tesla magnet, an external electrospray ion source and a Unix-based XMASS data system. The mass resolution is about 40,000 (50% valley definition).

Method 21 (preparative HPLC): instrument: Gilson Abimed HPLC; UV detector 210 nm; binary pump system; column: Reprosil ODS-A, 5 μm, 250×20 mm; eluent A: 0.2% trifluoroacetic acid in water, eluent B: acetonitrile; flow rate: 25 ml/min; column temperature 40° C.; 0-10 min 20% B, 10-15 min 80% B.

General Working Procedures

General Working Procedure 1 (Hydrolytic Sample Preparation for MALDI-MS)

The depsipeptide to be opened (e.g. lysobactin, 0.05 μmol) is first treated with a borate-hydrochloric acid buffer (Merck) pH 8 (250 μl) in a microvial. The mixture is left standing overnight, acetic acid (100 μl) is added and the sample is freeze-dried. The crude product is investigated by means of MALDI-MS sequencing without further purification steps.

General Working Procedure 2 (Edman^{0.5 and 1.5})

Phenyl isothiocyanate (50 mmol) is added dropwise to a solution of the N-terminal free peptide (0.3 mmol) in dry pyridine (30 ml) under an argon protective gas atmosphere. The reaction mixture is stirred at 37° C. (about 1 h) until the analytical HPLC check (Method 13) indicates adequate conversion (>95%). The reaction mixture is concentrated in a vacuum with temperature control (<40° C.) and then lyophilized.

General Working Procedure 3 (Edman^{1.0 and 2.0})

Under an argon protective gas atmosphere the peptide thiourea (0.2 mmol) is treated as a solid with dry trifluoroacetic acid with vigorous stirring and then stirred at 40° C. (about 20 min) until the analytical HPLC check indicates adequate conversion (>95%). The reaction mixture is rapidly concentrated in vacuo at room temperature (temperature control). In order to free the crude product of further trifluoroacetic acid, the crude product is taken up in dichloromethane and again freed of solvent in vacuo. This process is repeated a number of times with toluene (twice) and with dichloromethane (twice). Finally, the crude product is lyophilized.

Starting Compounds

### Example 1A

### D-Leucyl-N¹-{(3S,6S,12S,15S,18R,21S,24S,27S,28R)-6-[(1s)-2-amino-1-hydroxy-2-oxoethyl]-18-(3-{[amino(imino)methyl]amino}propyl)-12-[(1S)-1-hydroxyethyl]-3-(hydroxymethyl)-24-[(1R)-1-hydroxy-2-methylpropyl]-21-isobutyl-15-[(1S)-1-methylpropyl]-2,5,8,11,14,17,20,23,26-nonaoxo-28-phenyl-1-oxa-4,7,10,13,16,19,22,25-octaazacyclooctacosan-27-yl}-L-leucinamide bistrifluoroacetate(lysobactin)

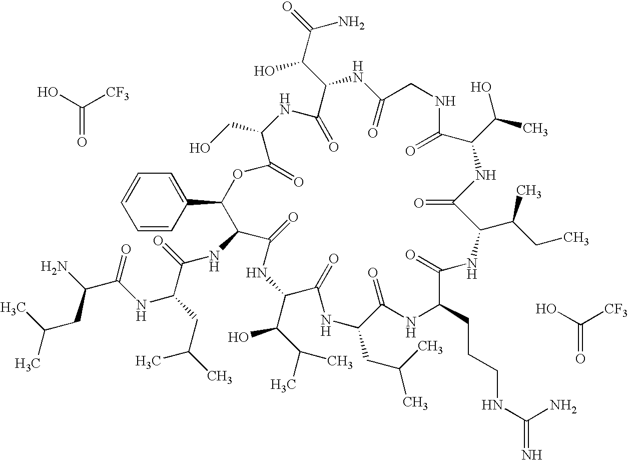

Fermentation:

Culture Medium:

YM: yeast-malt agar: D-glucose (4 g/l), yeast extract (4 g/l), malt extract (10 g/l), 1 litre of Lewatit water. Before sterilization (20 minutes at 121° C.), the pH is adjusted to 7.2.

HPM: mannitol (5.4 g/l), yeast extract (5 g/l), meat peptone (3 g/l).

Working preserve: The lyophilized strain (ATCC 53042) is grown in 50 ml of YM medium.

Flask fermentation: 150 ml of YM medium or 100 ml of RPM medium in a 1 l Erlenmeyer flask are inoculated with 2 ml of the working preserve and allowed to grow at 28° C. on a shaker at 240 rpm for 30-48 hours.

30 l fermentation: 300 ml of the flask fermentation (HPM medium) are used to inoculate a sterile 30 l nutrient medium solution (1 ml of antifoam SAG 5693/1). This culture is allowed to grow for 21 hours at 28° C., 300 rpm and aeration with sterile air of 0.3 vvm. The pH is kept constant at pH=7.2 using 1 M hydrochloric acid. In total, 880 ml of 1 M hydrochloric acid are added during the culturing period.

Main culture (200 l): 15×150 ml of YM medium in 1 l Erlenmeyer flasks are inoculated with 2 ml of the working preserve and allowed to grow on the shaker at 28° C. and 240 rpm for 48 hours. 2250 ml of this culture are used to inoculate a sterile 200 l nutrient medium solution (YM) (1 ml of antifoam SAG 5693/1) and it is allowed to grow for 18.5 hours at 28° C., 150 rpm and aeration with sterile air of 0.3 vvm.

Hourly samples (50 ml) are taken to check the course of the fermentation. 1 ml of methanol (0.5% trifluoroacetic acid) is added to 2 ml of this culture broth and the mixture is filtered through a 0.45 μm filter. 30 μl of this suspension are analysed by means of HPLC (Method 1 and Method 2).

After 18.5 hours, the culture broth of the main culture is separated into supernatant and sediment at 17000 rpm.

Isolation:

The supernatant (183 l) is adjusted to pH 6.5-7 using concentrated trifluoroacetic acid or sodium hydroxide solution and loaded onto a Lewapol column (OC 1064, 60 l contents). Elution is subsequently carried out with pure water, water/methanol 1:1 and subsequently with pure methanol (containing 0.1% trifluoroacetic acid). This organic phase is concentrated in vacuo to a residual aqueous residue of 11.5 l.

The residual aqueous phase is bound to silica gel C₁₈ and separated (MPLC, Biotage Flash 75, 75×30 cm, KP—C18-WP, 15-20 μm, flow: 30 ml; eluent: acetonitrile/water containing 0.1% trifluoroacetic acid; gradient: 10%, 15% and 40% acetonitrile). The 40% acetonitrile phase, which contains the main amount of Example 1A, is concentrated in vacuo and subsequently lyophilized (˜13 g). This mixture of solids is separated in 1.2 g portions, first on a preparative HPLC (Method 3), subsequently by gel filtration on Sephadex LH-20 (5×70 cm, acetonitrile/water 1:1, in each case containing 0.05% trifluoroacetic acid) and a further preparative HPLC (Method 4).

This process yields 2250 mg of Example 1A.

The sediment is taken up in 4 l of acetone/water 4:1, 2 kg of Celite are added, the mixture is adjusted to pH=6 using trifluoroacetic acid, stirred and centrifuged. The solvent is concentrated in vacuo and the residue is freeze-dried. The lyophilizate obtained (89.9 g) is taken up in methanol, filtered, concentrated and separated on silica gel (Method 5). Example 1A is then purified by gel filtration (Sephadex LH-20, 5×68 cm, water/acetonitrile 9:1 (containing 0.05% trifluoroacetic acid), flow: 2.7 ml/min, fraction size 13.5 ml) to give the pure substance.

This process yields 447 mg of Example 1A.

HPLC (Method 1): Rₜ=6.19 min

MS (ESIpos): m/z=1277 (M+H)⁺

¹H NMR (500.13 MHz, d₆-DMSO): δ=0.75 (d, 3H), 0.78 (d, 6H), 0.80 (t, 3H), 0.82 (d, 3H), 0.90 (d, 3H), 0.91 (d, 3H), 0.92 (d, 3H), 0.95 (d, 3H), 0.96 (d, 3H), 1.05 (m, 1H), 1.19 (d, 3H), 1.25 (m, 2H), 1.50 (m, 4H), 1.51 (m, 2H), 1.55 (m, 1H), 1.61 (m, 1H), 1.65 (m, 1H), 1.84 (m, 1H), 1.85 (m, 1H), 1.86 (m, 1H), 1.89 (m, 1H), 1.95 (m, 1H), 2.75 (m, 2H), 3.40 (m, 1H), 3.52 (m, 2H), 3.53 (dd, 1H), 3.64 (m, 2H), 3.66 (m, 1H), 3.68 (dd, 1H), 3.73 (m, 2H), 4.00 (dd, 1H), 4.02 (br., 1H), 4.13 (br., 1H), 4.32 (dd, 1H), 4.39 (t, 1H), 4.55 (m, 1H), 4.75 (dd, 1H), 5.19 (t, 1H), 5.29 (d, 1H), 5.30 (br., 1H), 5.58 (m, 2H), 6.68 (m, 3H), 6.89 (d, 1H), 6.93 (m, 3H), 6.94 (br., 1H), 6.98 (d, 1H), 7.12 (br., 1H), 7.20 (br., 2H), 7.23 (m, 2H), 7.42 (m, 2H), 7.54 (d, 1H), 7.58 (d, 1H), 8.32 (br., 1H), 9.18 (br., 1H), 9.20 (m, 2H), 9.50 (br., 1H).

¹³C-NMR (125.77 MHz, d₆-DMSO): δ=10.3, 15.3, 19.0, 19.2, 19.6, 20.0, 20.9, 22.0, 22.4, 23.0, 23.2, 24.3, 24.4, 25.0, 25.4, 26.0, 27.8, 30.9, 35.4, 39.5, 40.8, 40.9, 41.6, 44.1, 51.5, 52.7, 55.9, 56.2, 56.4, 57.9, 58.8, 60.2, 61.1, 62.6, 70.1, 71.6, 71.7, 75.5, 128.1, 128.6, 136.7, 156.8, 168.2, 170.1, 170.4, 171.2, 171.5, 171.9, 172.2, 172.4, 173.7.

The assignment of the signals was carried out according to the assignment described in the literature (T. Kato, H. Hinoo, Y. Terui, J. Antibiot., 1988, 61, 719-725).

### Example 2A

### N^{2.1}-[1-Methyl-3-oxobut-1-en-1-yl]-N^{7.ω},N^{7.ω′}-(pent[2]en[2]yl[4]ylidene)lysobactin

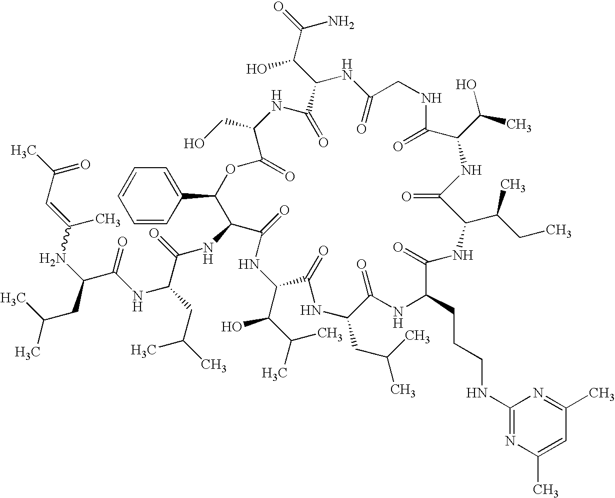

Powdered molecular sieve (4 Angstroms, 10 mg) and 2,4-pentanedione (200 equivalents, 0.2 ml, 2.0 mmol) are added to a solution of lysobactin bistrifluoroacetate (15 mg, 0.01 mmol) in pyridine (0.4 ml) in a pressure-resistant reaction vessel (size: 1 ml). The reaction mixture is first heated for 4 h at 80° C. and then at 90° C. until the HPLC chromatogram indicates complete conversion (about 12 h). The reaction mixture is filtered through a glass frit (pore size 2) while still hot, evaporated in vacuo and dried under high vacuum (12 h). The residue is purified by means of preparative HPLC (for example Method 17 without TFA). A solid (8 mg, 54% of th.) is obtained as the product.

LC-MS (Method 16): Rₜ=3.43 min;

MS (ESIpos.): m/z (%)=712 (100) [M+2H]²⁺.

MS (ESIneg.): m/z (%)=710 (100) [M−2H]²⁻.

### Example 3A

### N-(Anilinocarbonothioyl)-D-leucyl-N¹-{(3S,6S,12S,15S,18R,21S,24S,27S,28R)-6-[(1S)-2-amino-1-hydroxy-2-oxoethyl]-18-(3-{[amino(imino)methyl]amino}propyl)-12-[(1S)-1-hydroxyethyl]-3-(hydroxymethyl)-24-[(1R)-1-hydroxy-2-methylpropyl]-21-isobutyl-15-[(1S)-1-methylpropyl]-2,5,8,11,14,17,20,23,26-nonaoxo-28-phenyl-1-oxa-4,7,10,13,16,19,22,25-octaazacyclo-octacosan-27-yl}-L-leucinamide monotrifluoroacetate

### {N-(Anilinocarbonothioyl)lysobactin monotrifluoroacetate}

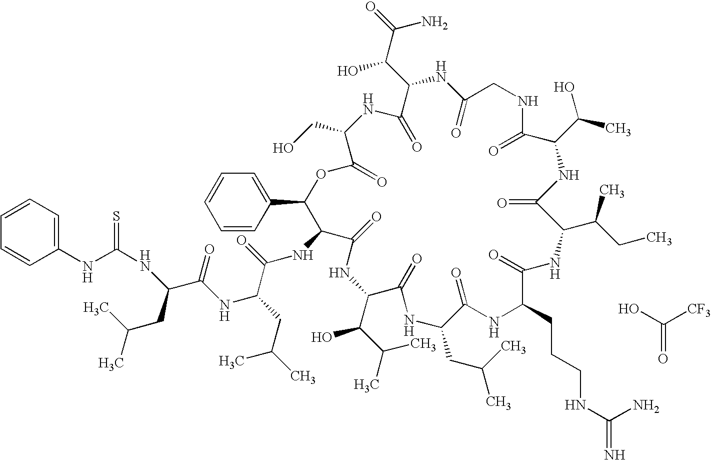

Lysobactin bistrifluoroacetate (500 mg, 0.33 mmol) (Example 1A) is reacted according to General working procedure 2. 600 mg (quant.) of product are obtained, which can be reacted further in unpurified form.

For further purification, the crude product can be gel-chromatographed (Method 6; methanol/0.1% acetic acid). The product-containing fractions are concentrated in vacuo at room temperature and then lyophilized. The product is obtained in 80% yield.

HPLC/UV-vis (Method 13): Rₜ=6.84 min,

λₘₐₓ (qualitative)=220 nm (s), 248 (m), 269 (m).

LC-MS (Method 11): Rₜ=2.64 min;

MS (ESIpos.): m/z (%)=706.5 (50) [M+2H]²⁺, 1412 (20) [M+H]⁺;

LC-MS (Method 12): Rₜ=4.95 min;

MS (ESIpos.): m/z (%)=1412 (100) [M+H]⁺.

### Example 4A

### N¹-{(3S,6S,12S,15S,18R,21S,24S,27S,28R)-6-[(1S)-2-Amino-1-hydroxy-2-oxoethyl]-18-(3-{[amino(imino)methyl]amino} propyl)-12-[(1S)-1-hydroxyethyl]-3-(hydroxymethyl)-24-[(1R)-1-hydroxy-2-methylpropyl]-21-isobutyl-I 5-[(1S)-1-methylpropyl]-2,5,8,11,14,17,20,23,26-nonaoxo-28-phenyl-1-oxa-4,7,10,13,16,19,22,25-octaazacyclo-octacosan-27-yl}-L-leucinamide bistrifluoroacetate

### {De-D-leucyllysobactin bistrifluoroacetate}

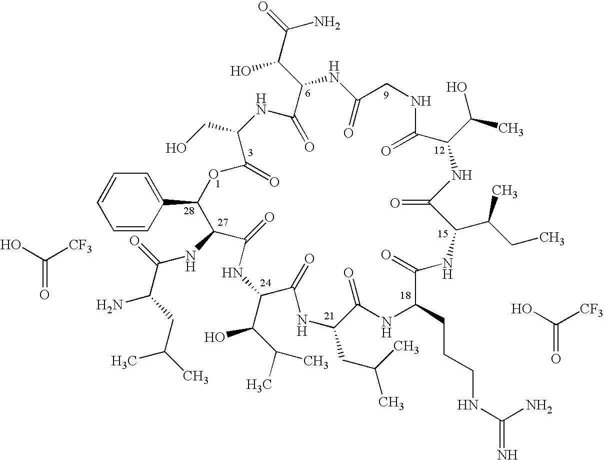

Thiourea (Example 3A) (300 mg, 0.2 mmol) is reacted according to General working procedure 3. The crude product is gel-chromatographed (Method 6; methanol/0.25% acetic acid) and subsequently fine purified by means of preparative HPLC (Method 8). 147 mg (65% of th.) of product are obtained.

HPLC/UV-vis (Method 13): Rₜ=4.96 min,

λₘₐₓ (qualitative)=220 nm (s), 255-270 (w).

LC-MS (Method 12): Rₜ=3.84 min;

MS (ESIpos.): m/z (%)=582.4 (100) [M+2H]²⁺, 1164 (20) [M+H]⁺.

FT-ICR-HR-MS (Method 20):

C₅₂H₈₈N₁₄O₁₆ [M+2H]²⁺ calc. 582.32459, fnd. 582.32460;

C₅₂H₈₇N₁₄NaO₁₆ [M+H+Na]²⁺ calc. 593.31556, fnd. 593.31564.

For amino acid sequence determination, an analytical sample of the product is hydrolysed according to General working procedure 1.

MALDI-MS (Method 19): m/z (%)=1181.7 (100) [M+H]⁺.

Alternative preparation process on a larger scale:

Example 1A (6.47 g, 4.30 mmol) is dissolved in pyridine (90 ml) under an argon atmosphere. Phenyl isothiocyanate (1.16 g, 8.60 mmol, 2 equivalents) is then added and the reaction mixture is stirred at 37° C. for 1 h. Subsequently, the solvent is distilled off on a rotary evaporator and the residue is dried overnight under an oil pump vacuum. The intermediate Example 2A is obtained in a crude yield of 6.60 g. The intermediate is reacted further without purification. To this end, Example 3A (6.60 g) is dissolved in trifluoroacetic acid (107 ml) under an argon atmosphere and stirred at room temperature for 30 min. The solution is then concentrated in vacuo on a rotary evaporator, briefly dried under an oil pump vacuum, taken up in methyl tert-butyl ether (250 ml) and stirred vigorously until a powdery amorphous solid results. This is collected by vacuum filtration and washed with methyl tert-butyl ether (200 ml), and then washed with dichloromethane (two times 100 ml). The solid is transferred to a flask and dried under an oil pump vacuum. Example 4A is obtained in a crude yield of 6.0 g (quant.). The product can be reacted without further purification.

### Example 5A

### N²-(Anilinocarbonothioyl)-N¹-{(3S,6S,12S,15S,18R,21S,24S,27S,28R)-6-[(1S)-2-amino-1-hydroxy-2-oxoethyl]-18-(3-{[amino(imino)methyl]amino}propyl)-12-[(1S)-1-hydroxyethyl]-3-(hydroxymethyl)-24-[(1R)-1-hydroxy-2-methylpropyl]-21-isobutyl-15-[(1S)-1-methylpropyl]-2,5,8,11,14,17,20,23,26-nonaoxo-28-phenyl-1-oxa-4,7,10,13,16,19,22,25-octaaza-cyclooctacosan-27-yl}-L-leucinamide monotrifluoroacetate

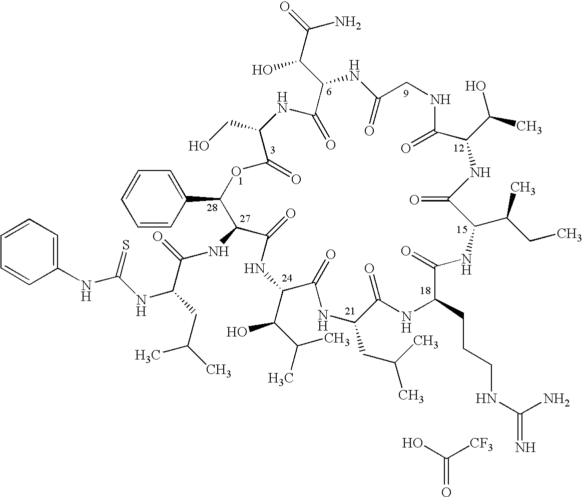

De-D-leucyllysobactin bistrifluoroacetate (Example 4A, 255 mg, 0.18 mmol) is reacted according to General working procedure 2. 322 mg (quant.) of product are obtained, which can be reacted further in unpurified form.

For further work-up, the crude product can be gel-chromatographed (Method 6; methanol/0.1% acetic acid). The product-containing fractions are concentrated in vacuo at room temperature and then lyophilized.

HPLC/UV-vis (Method 13): Rₜ=6.56 min,

λₘₐₓ (qualitative)=220 nm (s), 245 (m), 268 (m).

LC-MS (Method 12): Rₜ=4.85 min;

MS (ESIpos.): m/z (%)=1299 (100) [M+H]⁺.

### Example 6A

### (2S)-2-{(3S,6S,12S,15S,18R,21S,24S,27S,28R)-27-Amino-18-(3-{[amino(imino)methyl]amino}-propyl)-12-[(1S)-1-hydroxyethyl]-3-(hydroxymethyl)-24-[(1R)-1-hydroxy-2-methylpropyl]-21-isobutyl-15-[(1S)-1-methylpropyl]-2,5,8,11,14,17,20,23,26-nonaoxo-28-phenyl-1-oxa-4,7,10,13,16,19,22,25-octaazacyclooctacosan-6-yl}-2-hydroxyethanamide bistrifluoroacetate

### {De(1-D-leucyl-2-L-leucyl)lysobactin bistrifluoroacetate}

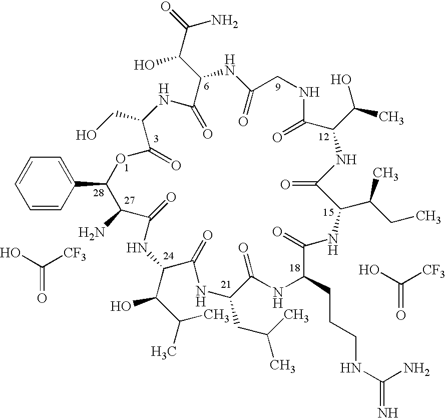

The thiourea (Example 5A, 66 mg, 34 μmol) is reacted according to General working procedure 3. The crude product can be pre-purified by rapid gel chromatography (Method 6; methanol/0.25% acetic acid). Preparative HPLC (Method 8 or Method 9 followed by subsequent double decomposition of the chromatographic product by addition of TFA (100 μmol)) yields 45 mg (75% of th.) of product.

HPLC/UV-vis (Method 13): Rₜ=4.71 min,

λₘₐₓ (qualitative)=220 nm (s), 255-270 (w).

LC-MS (Method 11): Rₜ=1.65 min;

MS (ESIpos.): m/z (%)=526 (100) [M+2H]²⁺, 1051 (15) [M+H]⁺.

Alternative Preparation Process on a Larger Scale:

Example compound 4A (6.47 g, 4.30 mmol) is dissolved in pyridine (92 ml) under an argon atmosphere. Phenyl isothiocyanate (8.75 g, 64.68 mmol, 15 equivalents) is then added and the reaction mixture is stirred at 37° C. for 1 hour. Subsequently, the solvent is distilled off on a rotary evaporator and the residue is dried overnight under an oil pump vacuum. Example 5A is obtained in a crude yield of 6.0 g. The intermediate is reacted further without purification. To this end, the crude Example 5A is dissolved in trifluoroacetic acid (82 ml) under an argon atmosphere and stirred at room temperature for 30 min. The solution is then concentrated in vacuo on a rotary evaporator, briefly dried under an oil pump vacuum, taken up in methyl tert-butyl ether (250 ml) and stirred vigorously until a powdery amorphous solid results. This is collected by vacuum filtration and washed with further methyl tert-butyl ether (200 ml), and then washed with two portions of 100 ml each of dichloromethane. The solid is transferred to a flask and dried under an oil pump vacuum. The title compound is obtained in a crude yield of 5.4 g (quant.). The product is further purified by preparative HPLC (Method 21). 1.79 g of the title compound (32% of th.) are obtained.

Exemplarily Embodiments

### Example 1

### N^{ω.6},N^{ω′.6}-(Pent[2]en[2]yl[4]ylidene)lysobactin trifluoroacetate

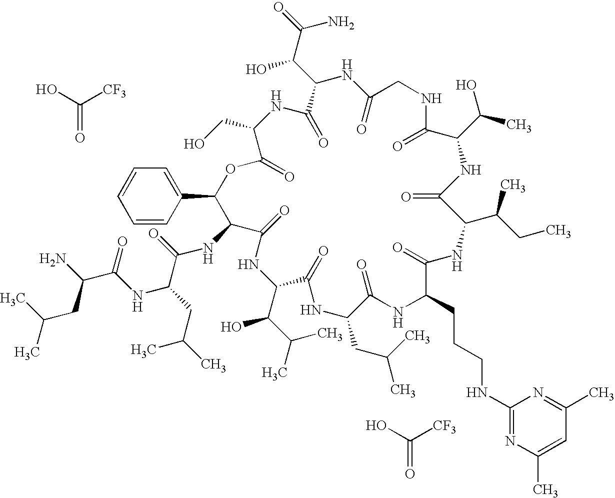

Powdered molecular sieve (4 Angstroms, 0.5 g) and 2,4-pentanedione (40 equivalents, 3.3 ml, 32.1 mmol) are added to a solution of lysobactin bistrifluoroacetate (2.0 g, 0.8 mmol) in pyridine (55 ml) in a three-necked flask equipped with a reflux condenser. The reaction mixture is first heated for 3.5 h at 85° C. and then at 110° C. until the HPLC chromatogram indicates complete conversion (about 4-8 h). The reaction mixture is filtered through a glass frit (pore size 2) while still hot, evaporated in vacuo and dried under high vacuum (12 h). The residue (1.9 g) is taken up in a mixture of acetonitrile (30 ml) and 0.5 N aqueous hydrochloric acid (40 ml) and stirred at room temperature until the HPLC chromatogram indicates complete conversion (about 0.4 h). The reaction mixture is concentrated in vacuo, frozen and freeze-dried. The cleavage product is purified by means of gel chromatography (Method 6, eluent methanol/acetic acid 99/1), whereby 1.5 g of crude product an obtained, which is subsequently fine purified by means of preparative HPLC (Method 7). 536 mg (46% of th.) of product are obtained.

HPLC/UV-vis (Method 9): Rₜ=5.9 min,

λₘₐₓ (qualitative)=220 nm (s), 310 (s).

LC-MS (Method 8): Rₜ=1.45 min;

MS (ESIpos.): m/z (%)=671 (100) [M+2H]²⁺, 1341 (10) [M+H]⁺.

MS (ESIneg.): m/z (%)=669 (80), 1339 (50) [M−H]⁻, 1385 [M−H+HCO₂H]⁻.

FT-ICR-HR-MS (Method 10): C₆₃H₁₀₃N₁₅O₁₇ [M+2H]²⁺ calc. 670.88227, fnd. 670.88169

TOF-HR-ESI-MS (Method 13): C₆₃H₁₀₂N₁₅O₁₇ [M+H]⁺ calc. 1340.7578, fnd. 1340.7552;

For the amino acid sequence determination, an analytical sample of the product is hydrolysed according to General working procedure 1.

MALDI-MS (Method 15): m/z (%)=1358.8 (100) [M+H]⁺.

The TFA content is determined via ¹⁹F-NMR (Method 18; calibration substance 1,4-dibromotetrafluorobenzene): calc. 14.5% by weight of TFA, fnd. 13.8% by weight of TFA.

### Example 2

### N^{ω.6},N^{ω′.6}-(1,1,1,5,5,5-Hexafluoropent[2]en[2]yl[4]ylidene)lysobactin

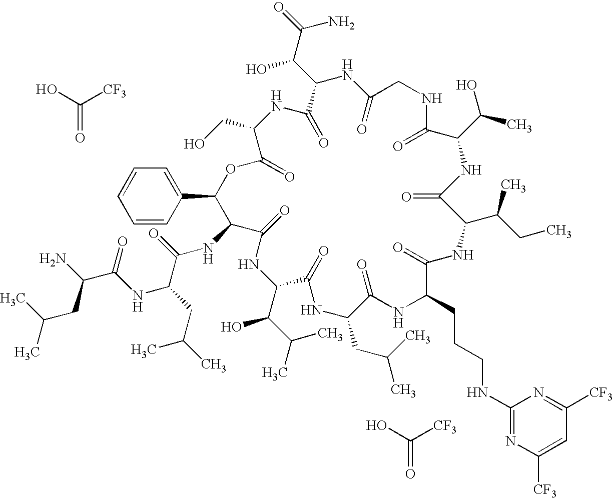

Powdered molecular sieve (4 Angstroms, 0.05 g) and 1,1,1,5,5,5-hexafluoro-2,4-pentanedione (10 equivalents, 70 μl, 480 μmol) are added to a solution of lysobactin bistrifluoroacetate (10.0 mg, 0.05 mmol) in pyridine (5 ml) in a three-necked flask equipped with a reflux condenser. The reaction mixture is first heated for 48 h at 85° C. and then at 95° C. until the HPLC chromatogram indicates complete conversion (about 12 h). The reaction mixture is filtered through a glass frit (pore size 2) while still hot, evaporated in vacuo and dried under high vacuum (12 h). The residue is taken up in a mixture of acetonitrile (3 ml) and 0.5 N aqueous hydrochloric acid (4 ml) and stirred at room temperature until the HPLC chromatogram indicates complete conversion (about 0.5 h). The reaction mixture is concentrated in vacuo, frozen and freeze-dried. The cleavage product is purified by means of preparative HPLC (Method 11). 3.5 mg (4.6% of th.) of product are obtained.

LC-MS (Method 12): Rₜ=2.68 min;

MS (ESIpos.): m/z (%)=725 (100) [M+2H]²⁺, 1449 (20) [M+H]⁺.

MS (ESIneg.): m/z (%)=687 (50), 1447 (100) [M−H]⁻, 1493 (15) [M−H+HCO₂H]⁻.

FT-ICR-HR-MS (Method 10): C₆₃H₉₅F₆N₁₅O₁₇ [M+2H]²⁺ calc. 724.85400, fnd. 724.85427

### Example 3

### N^{ω.6},N^{ω′.6}-(Pentane[2,4]diyl)lysobactin

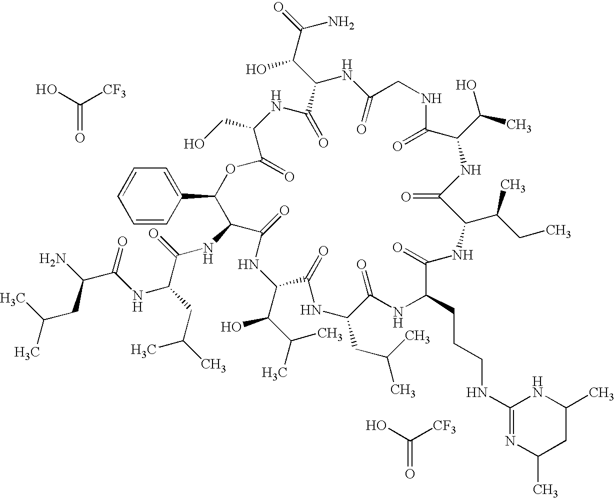

A mixture of N^{ω.6},N^{ω′.6}-(pent[2]en[2]yl[4]ylidene)lysobactin trifluoroacetate (205 mg, 0.14 mmol), 2-propanol (10 ml), water (10 ml), palladium on carbon (10%, 100 mg) and concentrated hydrochloric acid (1.8 ml) is hydrogenated under atmospheric pressure and at room temperature. The hydrogenation is terminated when the HPLC chromatogram indicates complete conversion (about 24 h). The reaction mixture is filtered through Celite (whereby it is washed several times with 2-propanol) and subsequently concentrated in vacuo. The crude product is purified by means of preparative HPLC (Method 14) and freeze-dried. A solid (52 mg, 25% of th.) is obtained as product.

HPLC/UV-vis (Method 9): Rₜ=6.0 min,

λₘₐₓ (qualitative)=220 nm (s), 260 (m).

LC-MS (Method 8): Rₜ=1.59 min;

MS (ESIpos.): m/z (%)=673 (100) [M+2H]²⁺, 1345 (10) [M+H]⁺.

MS (ESIneg.): m/z (%)=671 (80) [M−2H]²⁻, 1343 (40) [M−H]⁻, 1390 (100) [M−H+HCO₂H]⁻

TOF-HR-ESI-MS (Method 13): C₆₃H₁₀₆N₁₅O₁₇ [M+H]⁺ calc. 1344.7891, fnd. 1344.7867

### B. Evaluation of the Physiological Activity

The in vitro activity of the compounds of the invention can be shown in the following assays:

Determination of the Minimum Inhibitory Concentration (MIC):

The MIC is determined in the liquid dilution test in accordance with the NCCLS guidelines. Overnight cultures of Staphylococcus aureus 133, Entercococcus faecalis 27159, E. faecium 4147 and Streptococcus pneumoniae G9a are incubated with the described test substances in a 1:2 dilution series. The MIC determination is carried out with a cell count of 10⁵ microorganisms per ml in Isosensitest medium (Difco, Irvine/USA), with the exception of S. pneumoniae, which is tested in BHI broth (Difco, Irvine/USA) with 10% bovine serum at a cell count of 10⁶ microorganisms per ml. The cultures are incubated at 37° C. for 18-24 hours, S. pneumoniae in the presence of 10% CO₂.

The lowest substance concentration in each case at which no visible bacterial growth occurs any more is defined as the MIC. The MIC values are reported in μg/ml.

Representative in-vitro activity data for the compounds of the invention are shown in Table A:

TABLE A

| Example | MIC | MIC | MIC |
|---|---|---|---|
| No. | S. aureus 133 | S. pneumoniae | E. faecalis ICB 27159 |
| 3 | 0.5 | 0.5 | 2 |

The suitability of the compounds of the invention for the treatment of bacterial infections can be shown in the following animal model:

Systemic Infection with Staphylococcus Aureus 133:

Cells of S. aureus 133 are grown overnight in BHI broth (Oxoid, N.Y./USA). The overnight culture is diluted 1:100 in fresh BHI broth and incubated for 3 hours. The cells which are then in the logarithmic growth phase are centrifuged off and washed twice with buffered physiological saline. A cell suspension in saline is then adjusted photometrically to an extinction of 50. After a dilution step (1:15), this suspension is mixed 1:1 with a 10% mucin solution. 0.25 ml/20 g mouse of this infection solution is administered intraperitoneally (corresponding to 1×10⁶ microorganisms/mouse). The therapy takes place intraperitoneally or intravenously 30 minutes after infection. Female CFW1 mice are used for the infection experiment. The survival of the animals is recorded over a period of 6 days.

The properties of the compounds of the invention with respect to the renal tolerability can be shown in the following animal model:

Mouse Model for the Determination of Nephrotoxic Effects:

Nephrotoxic side effects of the nonadepsipeptides are analysed by histopathological examinations of the kidneys in mice and/or rats after multiple administration of a particular dose. For this, 5-6 animals are treated daily either intravenously (i.v.) or intraperitoneally (i.p.) with substances which are dissolved in an aqueous solution or with addition of Solutol. Nephrotoxic effects are determined by light-microscopical evaluation of haematoxilin and eosin (H&E) stained paraffin sections of the kidneys. A ‘periodic acid Schiff’ (PAS) reaction is optionally carried out for a better visualization of glycoproteins. Nephrotoxic effects are defined semiquantitatively for each animal as the degrees of severity of the tubular basophilia and degeneration/regeneration occurring (degrees of severity: 0=no effect; 1=minimal effect; 2=slight effect; 3=moderate effect; 4=severe lesions). The average degree of severity of the tubular degeneration/regeneration and the incidence (number of animals concerned) is calculated for each animal group or derivative. Kidney changes going beyond this, such as tubular dilatation and necrosis as well as the accumulation of necrotic materials, are likewise listed.

### C. Exemplary Embodiments of Pharmaceutical Compositions

The compounds of the invention can be converted into pharmaceutical preparations in the following ways:

Tablet:

Composition:

100 mg of the compound of Example 1, 50 mg of lactose (monohydrate), 50 mg of maize starch (native), 10 mg of polyvinylpyrolidone (PVP 25) (BASF, Ludwigshafen, Germany) and 2 mg of magnesium stearate.

Tablet weight 212 mg. Diameter 8 mm, radius of curvature 12 mm.

Preparation:

The mixture of active ingredient, lactose and starch is granulated with a 5% solution (m/m) of the PVP in water. The granules are dried and then mixed with the magnesium stearate for 5 min. This mixture is compressed using a conventional tablet press (see above for format of the tablet). A guideline for the compressive force used for compression is 15 kN.

Suspension which can be Administrated Orally:

Composition:

1000 mg of the compound of Example 1, 1000 mg of ethanol (96%), 400 mg of Rhodigel (xanthan gum from FMC, Pennsylvania, USA) and 99 g of water.

10 ml of oral suspension correspond to a single dose of 100 mg of the compound of the invention.

Preparation:

The Rhodigel is suspended in ethanol, and the active compound is added to the suspension. The water is added while stirring. The mixture is stirred for about 6h until the swelling of the Rhodigel is complete.

Solution which be can Administrated Intravenously:

Composition:

100-200 mg of the compound of Example 1, 15 g of polyethylene glycol 400 and 250 g of water for injection.

Preparation:

The compound of Example 1 is dissolved together with polyethylene glycol 400 in the water with stirring. The solution is sterilized by filtration (pore diameter 0.22 μm) and dispensed under aseptic conditions into heat-sterilized infusion bottles. The latter are closed with infusion stoppers and crimped caps.

What is claimed is:

1. A compound of formula 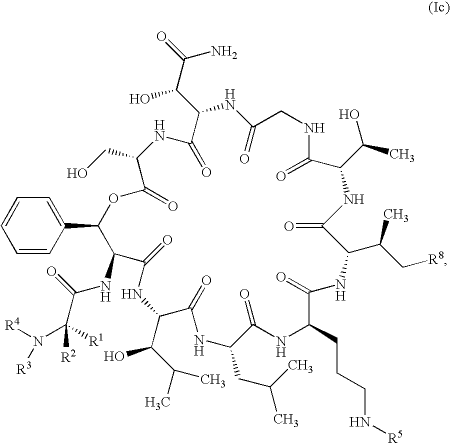
in which
R¹ represents hydrogen, C₁-C₆-alkyl, C₂-C₆-alkenyl, C₃-C₆-cycloalkyl or C₆-C₁₀-aryl,
whereby alkyl, alkenyl, cycloalkyl and aryl can be substituted with 0, 1, 2 or 3 substituents selected independently of one another from the group consisting of halogen, hydroxy, amino, cyano, trimethylsilyl, C₁-C₆-alkyl, C₁-C₆-alkoxy, benzyloxy, C₃-C₆-cycloalkyl, C₆-C₁₀-aryl, 5- to 7-membered heterocyclyl, 5- to 10-membered heteroaryl, C₁-C₆-alkylamino, C₆-C₁₀-arylamino, C₁-C₆-alkylcarbonylamino, C₆-C₁₀-arylcarbonylamino, C₁-C₆-alkylcarbonyl, C₁-C₆-alkoxycarbonyl, C₆-C₁₀-arylcarbonyl and benzyloxycarbonylamino,
wherein cycloalkyl, aryl, heterocyclyl and heteroaryl for their part can be substituted with 0, 1, 2 or 3 substituents selected independently of one another from the group consisting of halogen, hydroxy, amino, cyano, nitro, trifluoromethyl, C₁-C₆-alkyl, C₁-C₆-alkoxy, phenyl and 5- to 7-membered heterocyclyl,
R² represents hydrogen or C₁-C₄-alkyl,
R³ represents C₁-C₆-alkyl, C₃-C₆-cycloalkyl, 5- to 7-membered heterocyclyl, C₆-C₁₀-aryl, 5- or 6-membered heteroaryl, C₁-C₆-alkylcarbonyl, C₁-C₆-alkoxycarbonyl, C₃-C₆-cycloalkylcarbonyl, 5- to 7-membered heterocyclylcarbonyl, C₆-C₁₀-arylcarbonyl, 5- or 6-membered heteroarylcarbonyl or C₁-C₆-alkylaminocarbonyl,
whereby alkyl, cycloalkyl, heterocyclyl, aryl, heteroaryl, alkoxycarbonyl, cyclo-alkylcarbonyl, heterocyclylcarbonyl, arylcarbonyl, heteroarylcarbonyl and alkylaminocarbonyl can be substituted with 0, 1, 2 or 3 substituents selected independently of one another from the group consisting of halogen, hydroxy, amino, C₁-C₆-alkylamino and phenyl,
and
whereby alkylcarbonyl is substituted with a substituent amino or C₁-C₆-alkylamino, and
whereby alkylcarbonyl can be substituted with a further 0, 1 or 2 substituents selected independently of one another from the group consisting of halogen, hydroxy, trimethylsilyl, C₁-C₆-alkoxy, C₁-C₆-alkylthio, benzyloxy, C₃-C₆-cycloalkyl, phenyl, naphthyl, 5- to 10-membered heteroaryl, C₁-C₆-alkylcarbonylamino, C₁-C₆-alkoxycarbonylamino, C₆-C₁₀-arylcarbonylamino, C₆-C₁₀-arylcarbonyloxy, benzyloxycarbonyl and benzyloxycarbonylamino,
wherein phenyl and heteroaryl for their part can be substituted with 0, 1, 2 or 3 substituents selected independently of one another from the group consisting of halogen, hydroxy, nitro, C₁-C₆-alkyl, C₁-C₆-alkoxy and phenyl,
R⁴ represents hydrogen, C₁-C₄-alkyl, cyclopropyl or cyclopropylmethyl,
R⁵ represents a group of formula 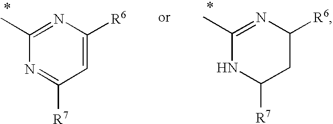
whereby
is the linkage site to the nitrogen atom,
R⁶ and R⁷ independently of one another represent C₁-C₆-alkyl or trifluoromethyl,
R⁸ represents hydrogen or methyl,
or one of its salts, its solvates or the solvates of its salts.

2. The compound of claim 1, corresponding to formula 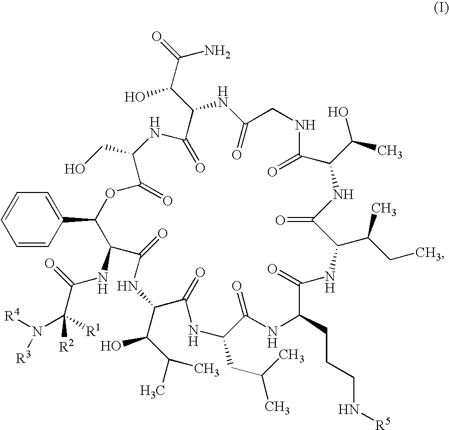
in which
R¹ represents hydrogen, C₁-C₆-alkyl, C₂-C₆-alkenyl, C₃-C₆-cycloalkyl or C₆-C₁₀-aryl,
whereby alkyl, alkenyl, cycloalkyl and aryl can be substituted with 0, 1, 2 or 3 substituents selected independently of one another from the group consisting of halogen, hydroxy, amino, cyano, trimethylsilyl, C₁-C₆-alkyl, C₁-C₆-alkoxy, benzyloxy, C₃-C₆-cycloalkyl, C₆-C₁₀-aryl, 5- to 7-membered heterocyclyl, 5- to 10-membered heteroaryl, C₁-C₆-alkylamino, C₆-C₁₀-arylamino, C₁-C₆-alkylcarbonylamino, C₆-C₁₀-arylcarbonylamino, C₁-C₆-alkylcarbonyl, C₁-C₆-alkoxycarbonyl, C₆-C₁₀-arylcarbonyl and benzyloxycarbonylamino,
wherein cycloalkyl, aryl, heterocyclyl and heteroaryl for their part can be substituted with 0, 1, 2 or 3 substituents selected independently of one another from the group consisting of halogen, hydroxy, amino, cyano, nitro, trifluoromethyl, C₁-C₆-alkyl, C₁-C₆-alkoxy, phenyl and 5- to 7-membered heterocyclyl,
R² represents hydrogen or C₁-C₄-alkyl,
R³ represents C₁-C₆-alkyl, C₃-C₆-cycloalkyl, 5- to 7-membered heterocyclyl, C₆-C₁₀-aryl, 5- or 6-membered heteroaryl, C₁-C₆-alkylcarbonyl, C₁-C₆-alkoxycarbonyl, C₃-C₆-cycloalkylcarbonyl, 5- to 7-membered heterocyclylcarbonyl, C₆-C₁₀-arylcarbonyl, 5- or 6-membered heteroarylcarbonyl or C₁-C₆-alkylaminocarbonyl,
whereby alkyl, cycloalkyl, heterocyclyl, aryl, heteroaryl, alkoxycarbonyl, cyclo-alkylcarbonyl, heterocyclylcarbonyl, arylcarbonyl, heteroarylcarbonyl and alkylaminocarbonyl can be substituted with 0, 1, 2 or 3 substituents selected independently of one another from the group consisting of halogen, hydroxy, amino, C₁-C₆-alkylamino and phenyl,
and
whereby alkylcarbonyl is substituted with a substituent amino or C₁-C₆-alkylamino, and
whereby alkylcarbonyl can be substituted with a further 0, 1 or 2 substituents selected independently of one another from the group consisting of halogen, hydroxy, trimethylsilyl, C₁-C₆-alkoxy, C₁-C₆-alkylthio, benzyloxy, C₃-C₆-cycloalkyl, phenyl, naphthyl, 5- to 10-membered heteroaryl, C₁-C₆-alkylcarbonylamino, C₁-C₆-alkoxycarbonylamino, C₆-C₁₀-arylcarbonylamino, C₆-C₁₀-arylcarbonyloxy, benzyloxycarbonyl and benzyloxycarbonylamino,
wherein phenyl and heteroaryl for their part can be substituted with 0, 1, 2 or 3 substituents selected independently of one another from the group consisting of halogen, hydroxy, nitro, C₁-C₆-alkyl, C₁-C₆-alkoxy and phenyl,
R⁴ represents hydrogen, C₁-C₄-alkyl, cyclopropyl or cyclopropylmethyl,
R⁵ represents a group of formula 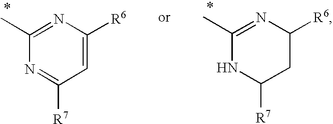
whereby
is the linkage site to the nitrogen atom,
R⁶ and R⁷ independently of one another represent C₁-C₆-alkyl or trifluoromethyl, or one of its salts, its solvates or of the solvates of its salts.

3. The compound of claim 2, whereby
R¹ represents 2-methylprop-1-yl, 2,2-dimethylprop-1-yl, 2,2-dimethylbut-1-yl, 1-trimethylsilylmethyl, 2-trimethylsilyleth-1-yl, 1-hydroxy-2-methylprop-1-yl, 1-hydroxy-2,2-dimethylprop-1-yl, 1-hydroxy-2,2-dimethylbut-1-yl, 1-hydroxy-2-ethyl-2-methylbut-1-yl, 1-hydroxy-2,2-diethylbut-1-yl, phenylmethyl, 1-hydroxy-1-phenylmethyl, 2-pyridylmethyl or 3-pyridylmethyl,
whereby 2-pyridylmethyl or 3-pyridylmethyl can be substituted with 0, 1, 2 or 3 substituents selected independently of one another from the group consisting of hydroxy, amino, trifluoromethyl, methyl, methoxy and morpholinyl,
R² represents hydrogen,
R³ represents 1-amino-3-methylbut-1-ylcarbonyl, 1-amino-3,3-dimethylbut-1-ylcarbonyl or 1-amino-2-trimethylsilyleth-1-ylcarbonyl,
R⁴ represents hydrogen,
R⁵ represents a group of formula 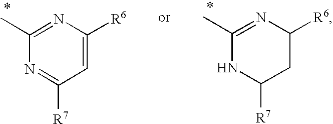
whereby
is the linkage site to the nitrogen atom,
R⁶ and R⁷ independently of one another represent C₁-C₆-alkyl or trifluoromethyl, or one of its salts, its solvates or the solvates of its salts.

4. The compound of claim 2, whereby
R¹ represents 2-methylprop-1-yl,
R² represents hydrogen,
R³ represents 1-amino-3-methylbut-1-ylcarbonyl,
R⁴ represents hydrogen,
R⁵ represents a group of formula 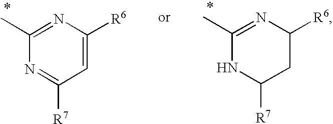
whereby
is the linkage site to the nitrogen atom,
R⁶ and R⁷ independently of one another represent C₁-C₆-alkyl, or one of its salts, its solvates or the solvates of its salts.

5. The compound of claim 2, whereby
R¹ represents 2,2-dimethylprop-1-yl,
R² represents hydrogen,
R³ represents 1-amino-3,3-dimethylbut-1-ylcarbonyl,
R⁴ represents hydrogen,
R⁵ represents a group of formula 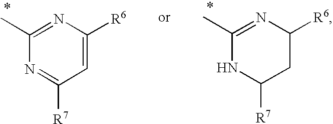
whereby
is the linkage site to the nitrogen atom,
R⁶ and R⁷ independently of one another represent C₁-C₆-alkyl, or one of its salts, its solvates or the solvates of its salts.

6. The compound of claim 2, whereby
R¹ represents 2,2-dimethylprop-1-yl, 1-trimethylsilylmethyl or 3-pyridylmethyl, whereby 3-pyridylmethyl can be substituted with a substituent trifluoromethyl,
R² represents hydrogen,
R³ represents 1-amino-3,3-dimethylbut-1-ylcarbonyl or 1-amino-2-trimethylsilyleth-1-ylcarbonyl,
R⁴ represents hydrogen,
R⁵ represents a group of formula 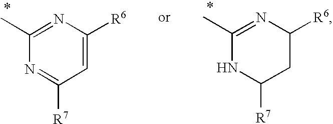
whereby
is the linkage site to the nitrogen atom,
R⁶ and R⁷ independently of one another represent C₁-C₆-alkyl or trifluoromethyl, or one of its salts, its solvates or the solvates of its salts.

7. A method for preparing a compound of formula (Ic) of claim 1, whereby according to method [A], a compound of formula 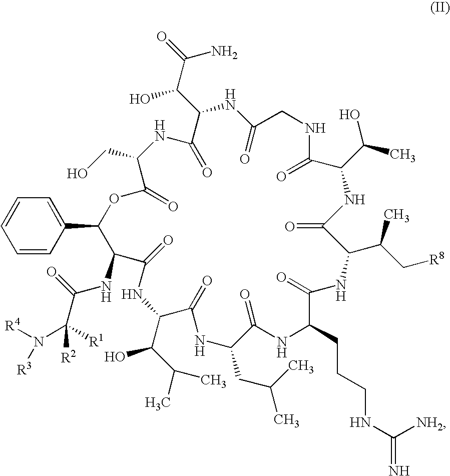
in which R¹, R², R³, R⁴ and R⁸ have the meaning indicated in claim 1, is reacted with a compound of formula 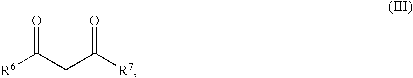
in which R⁶ and R⁷ have the meaning indicated in claim 1,
to give a compound of formula 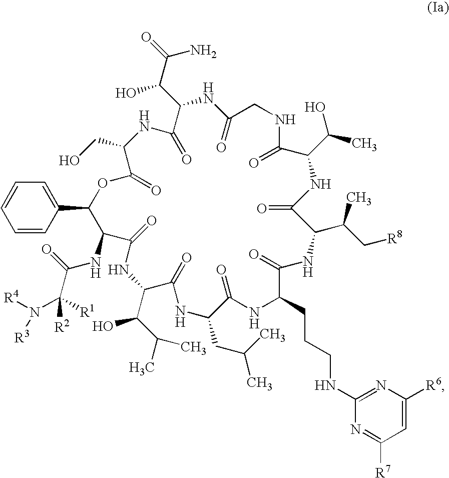
in which R¹, R², R³, R⁴, R⁶, R⁷ and R⁸ have the meaning indicated in claim 1, or
according to method [B], a compound of formula (Ia) is reacted with a reducing agent to give a compound of formula 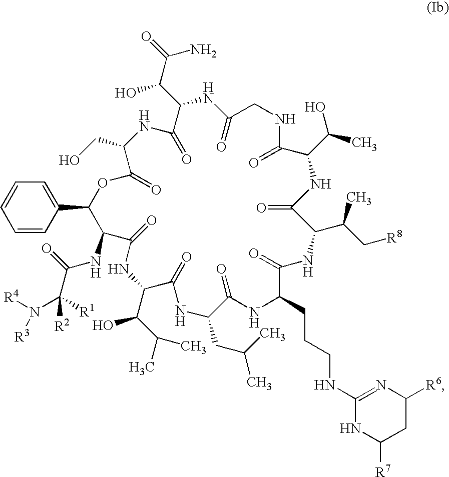
in which R¹, R², R³, R⁴, R⁶, R⁷ and R⁸ have the meaning indicated in claim 1.

8. The compound of claim 1 for the treatment of diseases.

9. The compound of claim 1 for the prophylaxis of diseases.

10. The compound of claim 1 for the treatment and prophylaxis of diseases.

11. A method for the production of a medicament for the treatment of diseases using a compound of claim 1.

12. A method for the production of a medicament for the prophylaxis of diseases using a compound of claim 1.

13. A method for the production of a medicament for the treatment and prophylaxis of diseases using a compound of claim 1.

14. A method for the production of a medicament for the treatment of bacterial infections using a compound of claim 1.

15. A method for the production of a medicament for the prophylaxis of bacterial infections. using a compound of claim 1

16. A method for the production of a medicament for the treatment and prophylaxis of bacterial infections. using a compound of claim 1

17. A medicament comprising a compound of claim 1 in combination with an inert, non-toxic, pharmaceutically acceptable excipient.

18. The medicament of claim 17 for the treatment of bacterial infections.

19. The medicament of claim 17 for the prophylaxis of bacterial infections.

20. The medicament of claim 17 for the treatment and prophylaxis of bacterial infections.

21. A method for controlling bacterial infections in humans and animals by administering an antibacterially effective amount of at least one compound of claim 1.

22. A method for controlling bacterial infections in humans and animals by administering an antibacterially effective amount of a medicament of claim 17.

23. A method for controlling bacterial infections in humans and animals by administering an antibacterially effective amount of a medicament obtained by the method of claim 11.

24. A method for controlling bacterial infections in humans and animals by administering an antibacterially effective amount of a medicament obtained by the method of claim 14.
